(19) United States
(12) Patent Application Publication
(10) Pub. No.: US 2006/0251574 A1
(43) Pub. Date: Nov. 9, 2006

(54) Cytotoxins and diagnostic imaging agents comprising hsp90 ligands

(76) Inventors: Adeela Kamal, San Diego, CA (US); Francis J. Burrows, Solana Beach, CA (US); Lin Zhang, San Diego, CA (US); Marcus Boehm, San Diego, CA (US)

Correspondence Address: WILSON SONSINI GOODRICH & ROSATI, 650 PAGE MILL ROAD, PALO ALTO, CA, 94304-1050, US

(21) Appl. No.: 10/538,687

(22) PCT Filed: Jun. 12, 2003
(86) PCT No.: PCT/US03/18776

§ 371 Date: Apr. 18, 2006

(30) Foreign Application Priority Data
Dec. 12, 2002 (WO) PCT/US02/39993

(51) Int. Cl. A61K 51/00; C07F 5/00; C07F 13/00

(52) U.S. Cl. 424/1.11; 534/11; 534/14; 540/456; 540/452; 534/15

(57) ABSTRACT

Cytotoxic compounds and diagnostic imaging agents comprising HSP90-ligands and methods of use thereof are described.

### RELATED APPLICATIONS

This application claims priority to Kamal et al., PCT/US02/39993, filed Dec. 12, 2002, entitled ASSAYS AND IMPLEMENTS FOR DETERMINING AND MODULATING HSP90 BINDING ACTIVITY, which claims priority to U.S. Provisional Patent Application Ser. No. 60/340,762, filed Dec. 12, 2001, and entitled ASSAYS FOR DETERMINING HSP90 BINDING ACTIVITY, each of which applications is herein incorporated by reference in its entirety.

### FIELD OF INVENTION

The invention relates generally to assays for assessing ligand binding and binding affinity, and more specifically to heat shock protein 90 (“HSP90”) binding assays.

### BACKGROUND

The following description includes information that may be useful in understanding the present invention. It is not an admission that any of the information provided herein is prior art or relevant to the presently claimed inventions, or that any publication specifically or implicitly referenced is prior art.

17-allylamino-geldanamycin (17-AAG) is a synthetic analog of geldanamycin (GDM). Both molecules belong to a broad class of antibiotic molecules known as ansamycins. GDM, as first isolated from the microorganism Streptomyces hygroscopicus, was originally identified as a potent inhibitor of certain kinases, and was later shown to act by stimulating kinase degradation, specifically by targeting “molecular chaperones,” e.g., heat shock protein 90s (HSP90s). Subsequently, various other ansamycins have demonstrated more or less such activity, with 17-AAG being among the most promising and the subject of intensive clinical studies currently being conducted by the National Cancer Institute (NCI). See, e.g., Federal Register, 66(129): 35443-35444; Erlichman et al., Proc. AACR (2001),42, abstract 4474.

HSP90s are ubiquitous chaperone proteins that are involved in folding, activation and assembly of a wide range of proteins, including key proteins involved in signal transduction, cell cycle control and transcriptional regulation. Researchers have reported that HSP90 chaperone proteins are associated with important signaling proteins, such as steroid homone receptors and protein kinases, including, e.g., Raf-1, EGFR, v-Src family kinases, Cdk4, and ErbB-2 (Buchner J., 1999, TIBBS, 24:136-141; Stepanova et al., 1996, Genes Dev. 10:1491-502; Dai, K. et al., 1996, J Biol. Chem, 271:22030-4).

Studies further indicate that certain co-chaperones, e.g., Hsp70, p60/Hop/Stil, Hip, Bag1, HSP40/Hdj2/Hsj 1, immunophilins, p23, and p50, may assist HSP90 in its function. See, e.g., Caplan, A., 1999, Trends in Cell Biol., 9: 262-68.

Ansamycin antibiotics, e.g., herbimycin A (HA), geldanamycin (GM), and 17-AAG are thought to exert their anticancerous effects by tight binding of the N-terminus pocket of HSP90 (Stebbins, C. et al., 1997, Cell, 89:239-250). This pocket is highly conserved and has weak homology to the ATP-binding site of DNA gyrase (Stebbins, C. et al., supra; Grenert, J. P. et al., 1997, J. Biol. Chem., 272:23843-50). Further, ATP and ADP have both been shown to bind this pocket with low affinity and to have weak ATPase activity (Proromou, C. et al., 1997, Cell, 90: 65-75; Panaretou, B. et al., 1998, EMBO J., 17: 4829-36). In vitro and in vivo studies have demonstrated that occupancy of this N-terminal pocket by ansamycins and other HSP90 inhibitors alters HSP90 function and inhibits protein folding. At high concentrations, ansamycins and other HSP90 inhibitors have been shown to prevent binding of protein substrates to HSP90 (Scheibel, T., H. et al., 1999, Proc. Natl. Acad. Sci. USA 96:1297-302; Schulte, T. W. et al., 1995, J. Bioi. Chem. 270:24585-8; Whitesell, L., et al., 1994, Proc. Natl. Acad. Sci. USA, 91:8324-8328). Ansamycins have also been demonstrated to inhibit the ATP-dependent release of chaperone-associated protein substrates (Schneider, c., L. et aI., 1996, Proc. Natl. Acad. Sci. USA, 93:14536-41; Sepp-Lorenzino et al., 1995, J Biol. Chem. 270:16580-16587). In either event, the substrates are degraded by a ubiquitin-dependent process in the proteasome (Schneider, C., L., supra; Sepp-Lorenzino, L., et al., 1995, J. Biol. Chem., 270:16580-16587; Whitesell, L. et al., 1994, Proc. Natl. Acad. Sci. USA, 91: 8324-8328).

This substrate destabilization occurs in tumor and non-transformed cells alike and has been shown to be especially effective on a subset of signaling regulators, e.g., Raf (Schulte, T. W. et ai., 1997, Biochem. Biophys. Res. Commun. 239:655-9; Schulte, T. W., et al., 1995, J. Biol. Chem. 270:24585-8), nuclear steroid receptors (Segnitz, B., and U. Gehring. 1997, J. Biol. Chem. 272:18694-18701; Smith, D. F. et al., 1995, Mol. Cell. Biol. 15:6804-12), v-src (Whitesell, L., et al., 1994, Proc. Natl. Acad. Sci. USA, 91:8324-8328) and certain transmembrane tyrosine kinases (Sepp-Lorenzino, L. et al., 1995, J. Biol. Chem. 270:16580-16587) such as EGF receptor (EGFR) and Her2/Neu (Hartmann, F., et al., 1997, Int. J. Cancer 70:221-9; Miller, P. et al., 1994, Cancer Res. 54:2724-2730; Mimnaugh, E. G., et al., 1996, J. Biol. Chem. 271:22796-801; Schnur, R. et al., 1995, J. Med. Chem. 38:3806-3812), CDK4, and mutant p53. Erlichman et al., Proc. AACR (2001), 42, abstract 4474. The ansamycin-induced loss of these proteins leads to the selective disruption of certain regulatory pathways and results in growth arrest at specific phases of the cell cycle (Muise-Heimericks, R. C. et al., 1998, J Biol. Chem. 273 :29864-72), and apoptosis, and/or differentiation of cells so treated (Vasilevskaya, A. et al., 1999, Cancer Res., 59:3935-40). Ansamycins and HSP90 ligands in general thus hold great promise for the treatment and/or prevention of many types of cancers and proliferative disorders.

In addition to anti-cancer and antitumorgenic activity, HSP90 inhibitors have also been implicated in a wide variety of other utilities, including use as anti-inflammation agents, anti-infectious disease agents, agents for treating autoimmunity, agents for treating ischemia, and agents useful in promoting nerve regeneration (See, e.g., Rosen et al., WO 02/09696; PCT/USO1/23640; Degranco et al., WO 99/51223; PCT/US99/07242; Gold, U.S. Pat. No. 6,210,974 B1). There are reports in the literature that fibrogenetic disorders including but not limited to scleroderma, polymyositis, systemic lupus, rheumatoid arthritis, liver cirrhosis, keloid formation, interstitial nephritis, and pulmonary fibrosis may be treatable. (Strehlow, WO 02/02123; PCT/USO1/20578). Still further HSP90 modulation, modulators, and uses thereof are reported in PCT/US03/04283, PCT/US02/35938, PCT/US02/16287, PCT/US02/06518, PCT/US98/09805, PCT/US00/09512, PCT/US01/09512, PCT/US01/23640, PCT/US01/46303, PCT/US01/46304, PCT/US02/06518, PCT/US02/29715, PCT/US02/35069, PCT/US02/35938, PCT/US02/39993, PCT/US03/10533, and PCT/US03/02686.

Recently, Nicchitta et aI., WO 01/72779 (PCT/USO1/09512), demonstrated that HSP90 can assume a different conformation upon heat shock and/or binding by the fluorophore bis-ANS. Specifically, Nicchitta et al. demonstrated that this induced conformation exhibits a higher affinity for certain HSP90 ligands than another conformation that predominates in normal cells.

A fundamental step in identifying and evaluating HSP90 ligands is to be able to conveniently assay their binding affinity for HSP90. A variety of nonisotopic procedures, e.g., colorimetric, enzymatic, and densitometric, afford sufficient sensitivity in other contexts where they are preferred over isotopic procedures for health and disposal reasons, and Chiosis et al., Chemistry and Biology, 8:289-299 (2001), recently described a procedure for evaluating HSP90 ligand ability. The Chiosis procedure, however, is cumbersome and time-consuming from the standpoint of requiring gels to be run, blotted, and probed with antibody. The Chiosis assay is further limited in its ability to conveniently support high-throughput screening. Further, it appears that Chiosis employed a standard form of HSP90 that is characteristic of normal, healthy cells.

Needed are new diagnostic agents and therapies based on selective targeting of the HSP90 conformation that typifies cancerous, tumorous and infected cells.

### SUMMARY OF THE INVENTION

The aspects and embodiments of the present invention build on those featured in PCT/US02/39993, which reported the identification of high-affinity conformations of HSP90 in cancer cells and uses thereof in HSP90 binding assays and HSP90 inhibitor identification. This application features the use of such high-affinity agents in vitro and/or in vivo to selectively target cells containing such high affinity forms for, on the one hand, diagnostic and prognostic assessment, and on the other hand for selective therapeutics and/or prophylactic protection against diseases and afflictions mediated and/or characterized by high affinity forms of HSP90. Applicants' report herein of the selective accumulation and persistence of HSP90 inhibitors in tumor and cancer cells relative to normal cells suggests that these agents will find excellent use as both diagnostic and therapeutic agents.

Thus, in a first aspect the invention features a diagnostic or cytotoxic composition having an HSP90 inhibitor functionalized with an additional moiety that affords imaging and/or cytotoxin properties. The HSP90 ligand preferably includes a member selected from the group consisting of purines, ansamycins, radicicol, zearalanols, ATP analogs, indoles, chalcones, and benzimidazoles. In some preferred embodiments, the inhibitor is a benzoquinoid ansamycin, preferably derivatized at position 17 with the cytotoxic and/or imaging agent/moiety. One embodiment for such a compound may be generically illustrated by the following formula, 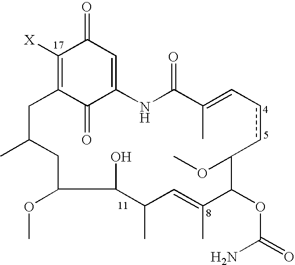
wherein X comprises the functional moiety, and wherein positions 4 and 5 are optionally both hydroxylated.

In some preferred embodiments, the functional moiety comprises a radioisotope/radionuclide, e.g., one selected from the group consisting of Iodene¹²⁵, Iodene¹³¹, Technitium^{99m}, Indium¹¹¹, Rhenium¹⁸⁸, Gallium⁶⁷, Copper⁶⁷, Yttrium⁹⁰, and Astatine²¹¹, ¹⁸F, ¹¹C, ¹³N, ¹²³I, 124I, ¹²⁵I, ¹³¹I, ²¹³Bi and ¹⁵O.; it is expected that these will find use not only as cytotoxic agents but also as imaging agents. In other preferred embodiments, the functional moiety comprises a mustin or mustard-type molecule. Illustrative species of these two embodiments include compounds of formulas

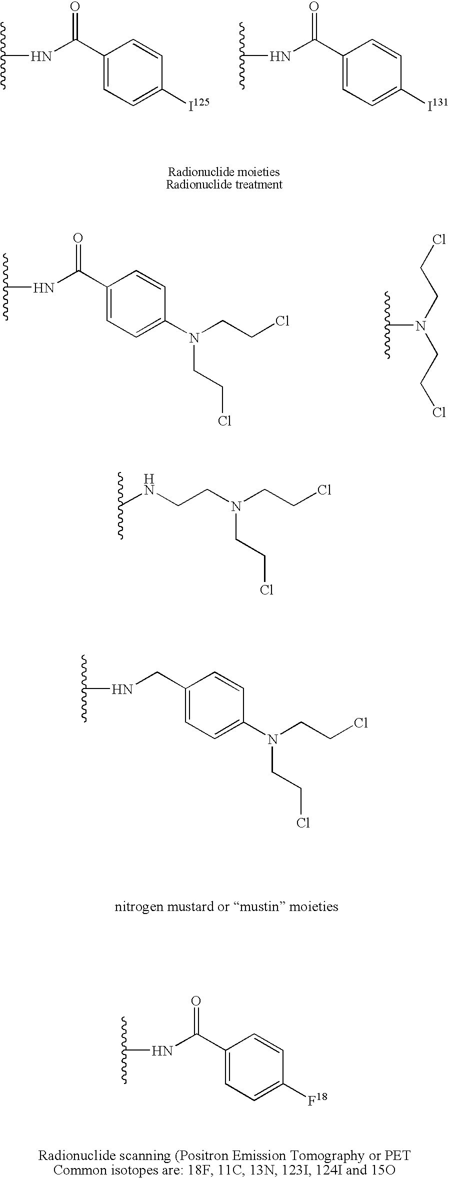

In addition or as an alternative to the foregoing, the functional moieties of the invention may also include a member selected from the group consisting of antibodies, recombinant products, small molecules, antineoplastic agents, herceptin, taxol, taxanes and taxane derivatives, gleevec, alkylating agents, anti-metabilites; epidophyllotoxin; an antineoplastic enzyme; a topoisomerase inhibitor; procarbazine; mitoxantrone; platinum coordination complexes; biological response modifiers/growth inhibitors; hormonal/anti-hormonal therapeutic agents and haematopoietic growth factors, anthracycline drugs, vinca drugs, mitomycins, bleomycins, cytotoxic nucleosides, tepothilones, discodermolide, pteridine drugs, diynenes, podophyllotoxins, carminomycin, daunorubicin, aminopterin, methotrexate, methopterin, dichloromethotrexate, mitomycin C, porfiromycin, 5-fluorouracil, 6-mercaptopurine, gemcitabine, cytosine arabinoside, podophyllotoxin, podo-phyllotoxin derivatives, etoposide, etoposide phosphate or teniposide, melphalan, vinblastine, vincristine, leurosidine, vindesine, leurosine, paclitaxel, estramustine, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins.

In another aspect, the invention features a method of treating or preventing an HSP90-mediated disease by administering to a subject a pharmaceutically effective amount of a composition according to the preceding aspect and embodiments. The disease can be a neoplastic disease or one established of fostered by a pathogen, e.g., a virus, bacteria, or fungus. In some preferred embodiments, the disease is a cancer or tumor, preferably one selected from the group consisting of melanoma, breast, lung, or prostate cancer or tumor. In some embodiments, the diseased cells express supra-normal levels of Her-2 transcript, protein, or HSP90 client proteins. The treatment may be administered, e.g., orally, topically, subcutaneously, intramuscularly, intratumorally, parenterally or intravenously.

In some embodiments, the administration is made to removed tissues or cells of an organism (ex vivo). In other embodiments, the administration is made to the living organism itself (in vivo), which may entail an in situ administration directly to the site of a tumor and/or a general systemic administration. Preferably the subject is a mammal, more preferably a human. In some embodiments, the treatment is part of a chemotherapy regimen.

In another aspect, the invention features a method of diagnosing or monitoring the progression or regression of an HSP90-mediated disease by administering to the cells of a subject having or suspected of having an HSP90-mediated disease a composition according to the first aspect, and then evaluating those cells for the presence of underlying compound. Because the compound selectively accumulates and persists in diseased cells, it is easy to image such cells using, e.g., the well-known procedure of positron emission tomography (PET) or equivalent imaging technology. The images created can, e.g., assist surgeons in identifying more precisely the location of tumorous or cancerous cells so as to excise or biopsy them. Alternatively, the imaging can be used to help diagnose disease or monitor its progression, regression, or the effect of treatment thereon. In the case of infectious diseases, normal cells and infected cells or tissues can be compared for the relative presence and/or concentration of the compounds of the invention as a measure of the disease state, or as a measure of therapy progress.

Additional advantages, aspects, and embodiments of the invention will be apparent from the figures, the detailed description, and claims to follow.

### BRIEF DESCRIPTION OF THE DRAWINGS

FIG. 1 shows competitive binding of geldanamycin and biotinylated geldanamycin for HSP90.

FIG. 2 shows competitive binding of 17-allyl amino geldanamycin (17-AAG) and biotinylated geldanamycin for HSP90.

FIG. 3 shows competitive binding of free geldanamycin, 17-AAG, and biotinylated geldanamycin for HSP90.

FIG. 4 shows that 17-AAG (CF7) has a higher apparent binding affinity for HSP90 from tumor cells (BT474) than normal cells (fibroblasts, RPTEC) or purified HSP90 alone, as determined using methods described herein.

FIG. 5 shows that 17-AAG (CF7) has a higher apparent binding affinity for HSP90 from the specific high Her2 expressing cells, SKOY-3, SKBR-3, and N87, than from normal cells, heat-shocked HSP90, or bis-ANS treated HSP90.

FIG. 6 shows the results of various test compounds used in certain assay embodiments of the invention. The cell line used was MCF7. Synthesis and use of the modulators shown is described in PCT/US02/29715.

FIG. 7 shows the pharmacokinetic profile of CF237 in a subcutaneous human tumor xenograft and in the liver and intestine of the host mice.

FIG. 8 shows the pharmacokinetic profile of CF237 in a subcutaneous human tumor xenograft and in the blood of the host mice.

FIG. 9 shows the in vitro cellular drug uptake kinetics of CF237 in a human tumor breast tumor cell line and in normal human epithelial and endothelial cells.

### DETAILED DESCRIPTION OF THE INVENTION

### DEFINITIONS

Compounds of the invention may embrace pharmaceutical salts of the underlying compounds. When a compound is named or displayed, it will be understood that pharmaceutically acceptable salts of such compound are also subsumed within the definition or structure.

A “pharmaceutically acceptable salt” may be prepared for any compound of the appropriate aspect of the invention having a functionality capable of forming a salt, for example an acid or base functionality. Pharmaceutically acceptable salts may be derived from organic or inorganic acids and bases. Compounds of the invention that contain one or more basic functional groups, e.g., amino or alkylamino, are capable of forming pharmaceutically acceptable salts with pharmaceutically acceptable organic and inorganic acids. These salts can be prepared in situ during the final isolation and purification of the compounds of the invention, or by separately reacting a purified compound of the invention in its free base form with a suitable organic or inorganic acid, and isolating the salt thus formed. Examples of suitable acids include hydrochloric, hydrobromic, sulfuric, nitric, perchloric, fumaric, maleic, phosphoric, glycolic, gluconic, lactic, salicylic, succinic, toluene-p-sulfonic, tartaric, acetic, citric, methanesulfonic, fonnic, benzoic, malonic, naphthalene-2-sulfonic, benzenesulfonic, 1,2 ethanesulfonic acid (edisylate), galactosyl-d-gluconic acid, and the like. Other acids, such as oxalic acid, while not themselves pharmaceutically acceptable, may be employed in the preparation of pharmaceutically acceptable acid addition salts. See, e.g., Berge et al. “Pharmaceutical Salts”, J. Pharm. Sci. 66:1-19 (1977).

Compounds of the present invention that contain one or more acidic functional groups are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the. hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Illustrative examples of some of the bases that can be used include sodium hydroxide, potassium hydroxide, choline hydroxide, sodium carbonate, and the like. Representative organic amines useful for the formation of base addition salts include ethyl amine, diethylamine, ethylenediamine, ethanolamine, diethanolamine, piperazine and the like. See, for example, Berge et al., supra.

A “pharmacological composition” refers to a mixture of one or more of the compounds described herein or pharmaceutically acceptable salts thereof, with other chemical components, such as pharmaceutically acceptable carriers and/or excipients. The purpose of a pharmacological composition is to facilitate administration of a compound to an organism.

The phrase “pharmaceutically acceptable carrier” as used herein means a pharmaceutically-acceptable material, composition or vehicle, such as a liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyllaurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. A physiologically acceptable carrier should not cause significant irritation to an organism and does not abrogate the biological activity and properties of the administered compound.

An “excipient” refers to an inert substance added to a pharmacological composition to further facilitate administration of a compound. Examples of excipients include but are not limited to calcium carbonate, calcium phosphate, various sugars and types of starch, cellulose derivatives, gelatin, vegetable oils and polyethylene glycols.

A “pharmaceutically effective amount” means an amount which is capable of providing a therapeutic and/or prophylactic effect. The specific dose of compound administered according to this invention to obtain therapeutic and/or prophylactic effect will, of course, be determined by the particular circumstances surrounding the case, including, for example, the specific compound administered, the route of administration, the condition being treated, and the individual being treated. A typical daily dose (administered in single or divided doses) will contain a dosage level of from about 0.01 mg/kg to about 50-100 mg/kg of body weight of an active compound of the invention. Preferred daily doses generally will be from about 0.05 mg/kg to about 20 mg/kg and ideally from about 0.1 mg/kg to about 10 mg/kg. Factors such as clearance rate and half-life and maximum tolerated dose (MTD) have yet to be determined but one of ordinary skill in the art can determine these using standard procedures.

In some method embodiments, the preferred therapeutic effect is the inhibition, to some extent, of the growth of cells characteristic of a proliferative disorder, e.g., breast cancer. A therapeutic effect will also normally, but need not, relieve to some extent one or more of the symptoms other than cell growth or size of cell mass. A therapeutic effect may include, for example, one or more of 1) a reduction in the number of cells; 2) a reduction in cell size; 3) inhibition (i.e., slowing to some extent, preferably stopping) of cell infiltration into peripheral organs, e.g., in the instance of cancer metastasis; 3) inhibition (i.e., slowing to some extent, preferably stopping) of tumor metastasis; 4) inhibition, to some extent, of cell growth; and/or 5) relieving to some extent one or more of the symptoms associated with the disorder.

In some preferred embodiments of the invention, the “IC50” value of a compound of the invention is greater for normal cells than for cells exhibiting a proliferative disorder, e.g., breast cancer cells. The value depends on the assay used.

By a “standard” is meant a positive or negative control. A negative control in the context of HER-2 expression levels is, e.g., a sample possessing an amount of HER-2 protein that correlates with a normal cell. A negative control may also include a sample that contains no HER-2 protein. By contrast, a positive control does contain HER-2 protein, preferably of an amount that correlates with over-expression as found in proliferative disorders, e.g., breast cancers. The controls may be from cell or tissue samples, or else contain purified ligand (or absent ligand), immobilized or otherwise. In some embodiments, one or more of the controls may be in the form of a diagnostic “dipstick.”

By “selectively targeting” is meant affecting one type of cell to a greater extent than another, e.g., in the case of cells with high as opposed to relatively low or normal Her-2 levels.

HSP90 Ligands

HSP90 ligands useful in the compositions and methods of the invention include, e.g., purines, ansamycins, radicicol, zearalanols, ATP analogs, indoles, chalcones, and benzimidazoles, which compounds are well-known in the art. Preferred are ansamycins, which are known to inhibit HSP90 function. The term “ansamycin” as used herein is well-known in the art and refers to a broad class of structures characterized by aliphatic rings of various length and constitution bridging opposite ends of aromatic ring structures and their reduced equivalents. Subsumed within this broad class is the sub-class, benzoquinone ansamycins. A “benzoquinone ansamycinin” as used herein possesses a benzoquinone as the aromatic ring structure and includes any benzoquinone ansamycin known in the art having an alkoxy moiety on the benzoquinone portion of the molecule, preferably a methoxy, and preferably at the 17 position, that can be replaced by a nucleophile. The result of the reaction is the formation of a “benzoquinone ansamycin derivative.” Ansamycins further include dimers and pharmaceutically acceptable salts of the ansamyin compound. Ansamycins may be synthetic, naturally-occurring, or a combination of the two, i.e., “semi-synthetic.” Exemplary ansamycins useful in the processes of the invention and their methods of preparation include but are not limited to those described, e.g., in U.S. Pat. No. 3,595,955 (describing the preparation of geldanamycin), U.S. Pat. Nos. 4,261,989, 5,387,584, and 5,932,566. Geldanamycin is also commercially available, e.g., from CN Biosciences, an affiliate of Merck headquartered in San Diego, Calif., USA (cat. no. 345805). The biochemical purification of 4,5-Dihydrogeldanamycin and its hydroquinone from cultures of Streptomyces hygroscopicus (ATCC 55256) are described Cullen et al. in International Application Number PCT/US92/10189 (WO 93/14215), assigned to Pfizer Inc. An alternative method of synthesis for 4,5-Dihydrogeldanamycin by catalytic hydrogenation of geldanamycin is also known. See, e.g., Progress in the Chemistry of Organic Natural Products, Chemistry a/the Ansamycin Antibiotics, 33 1976, p. 278. Applicants recently described the preparation of numerous other ansamycin compounds in commonly owned co-pending applications PCT/US03/04283 and PCT/US02/29715, including the dimer CF237 which appears in one of the examples below.

HSP90s

HSP90 proteins are ubiquitous cellular proteins that are highly conserved in nature. The term “an HSP90” or “an HSP90 member” as claimed herein includes but is not limited to the following: NCBI accession #'s P07900 and XM 004515 (human α and β HSP90 respectively), P11499 (mouse), AAB2369 (rat), P46633 (chinese hamster), JC1468 (chicken), AAF69019 (flesh fly), AAC21566 (zebrafish), AAD30275 (salmon), 002075 (pig), NP 015084 (yeast), and CAC29071 (frog). Further included in the definition are any variations of such proteins that may exist in nature or that are man-made. All are expected to have more or less utility in connection with the methods, assays, and ligands of the invention, e.g., in identifying and/or quantifying binding affinities of various HSP90 ligands, and thereby identifying and/or prioritizing new drug candidates for clinical trials. One aspect of the invention exploits Applicants' finding that cancer and tumor cells possess a more sensitive, high affinity, form of HSP90s than do normal cells. Ligands bind HSP90s found in cancer or tumor cells much more avidly despite the protein itself having an identical amino acid constituency. Without being limiting of the invention, this is thought to be a consequence of a different tertiary or quarternary configuration of the protein that is present in such cells, possibly being afforded by co-chaperone proteins or client proteins that are bound to the HSP90 to make it behave as such.

The following discussion sections concerning labeling and solid supports and high throughput screening are borrowed largely from U.S. Pat. Nos. 6,203,989, 6,153,442, 6,096,508, 5,846,537, and 5,585,241. The procedures described therein and below can be assimilated, adapted, and/or otherwise implemented in furtherance of the novel and unobvious features of the present invention.

Labels and Labeling

The following discussion sections concerning labeling and solid supports and high throughput screening are borrowed largely from U.S. Pat. Nos. 6,203,989, 6,153,442, 6,096,508, 5,846,537, and 5,585,241. The procedures described therein and below can be assimilated, adapted, and/or otherwise implemented in furtherance of the novel and unobvious features of various aspects and embodiments of the present invention.

Biotin:(Strept)Avidin Labels. Some preferred embodiments of the invention exploit the natural high affinity of streptavidin for biotin. Streptavidin is related to avidin, a 67 kilodalton (kD) glycoprotein found in e˜g whites and which has an exceptionally high binding affinity (K.sub.d=10.sup.−15 M) for biotin. Avidin consists of four subunits, each capable of binding one biotin molecule. Streptavidin is produced by Streptomyces avidinii and shares significant conformation and amino acid composition with avidin, as well as high affinity for biotin and stability. Streptavidin is also not glycosylated and reportedly exhibits less non-specific binding than avidin, making it the superior choice of the two for most biotin-based applications.

Biotin, a member of the B-complex vitamins, is found naturally and is essential for amino acid and fatty acid degradation, gluconeogenesis, and fatty acid synthesis. The binding interactions between biotin and the biotin binding site of avidin and streptavidin are the result of non covalent hydrogen bonding and van der Waals interactions between biotin and avidin, together with the ordering of surface polypeptide loops that bury the biotin in the protein interior. Biotin has previously been chemically or enzymatically coupled extensively to probe biomolecules in ways that minimize interference with target recognition, and the results described herein for geldanamycin provide further examples of how this may be done.

Reagents for labeling streptavidin or avidin with a fluorescent tag are commercially available. For example, the reagents, 5(6)-Carboxyfluorescein-N-hydroxysuccinimide ester (FLUOS), 7-amino4-methyl-coumarin-3-acetic acid-N′-hydroxysuccinimide ester (AMCA, activated) and fluorescein isothiocyanate (FITC) are available from Boehringer Mannheim, Indianapolis, Ind., USA. Methods for fluorescently labeling proteins with fluorescent labels, and methods for detection of the fluorescent labels, are described in Howard, G., Labeling Proteins with Fluorochromes, in “Methods in Nonradioactive Detection,”, G, Howard; Ed., Appleton and Lange, Norwalk, Conn., USA. 1993, pp. 39-68, the disclosure of which is incorporated herein. Additionally, there are a variety of commercially available streptavidin- and avidin-labeled molecules. Examples include streptavidin-gold, streptavidin-fluorochrome, streptavidin-AMCA, streptavidin-fluorescein, streptavidin-phycoerythrin (STPE), streptavidin-sulforhodamine 101, avidin-FITC and avidin-Texas red.RTM:, which are commercially available from Boehringer Mannheim, Indianapolis, Ind., USA. A working example of the use of streptavidin-phycoerythrin in the methods and reagents of some aspects and embodiments of the invention is described below.

In addition to the biotin-steptavidin system described above, other labels or label complexes can also be used to generate a detectable signal to relate the amount of bound and/or unbound label. The label can be any molecule that produces or can be induced to produce a signal, and may be, for example, a fluorescer, radio-label, enzyme, dense element such as gold, chemiluminescer or photosensitizer. Thus, the signal, depending on the label embodiment, can be detected and/or measured by detecting enzyme activity, luminescence, light absorbance or radioactivity as the case may be. In some applications, nonradioactive applications are preferred.

Specific labels that can be used illustratively include, e.g., enzymes such as alkaline phosphatase, glucose-6-phosphate dehydrogenase (“G6PDH”) and horseradish peroxidase;.dyes; fluorescers such as fluorescein, isothiocyanate, rhodamine, phycoerythrin, phycocyanin, allophycocyanin, o-phthaldehyde, and fluorescamine; chemiluminescers such as isoluminol; sensitizers; coenzymes; enzyme substrates; radiolabels such as .sup.125 I, .sup.131 I, .sup.14 C, .sup.3 H, .sup.57 Co and .sup.75 Se; particles such as latex, or carbon particles; metals; crystallite; liposomes; cells, photoactivatable compounds etc., which may be further labeled with a dye, catalyst or other detectable group. Other suitable enzymes and coenzymes are disclosed in Litman, et al., U.S. Pat. No. 4,275,149, columns 19-28, and Boguslaski, et al., U.S. Pat. No. 4,318,980, columns 10-14. Suitable fluorescers and chemiluminescers are disclosed in Litman, et al., U.S. Pat. No. 4,275,149, at columns 30 and 31. Photoactivatable compounds include but are not limited to those containing reactive enediyne and terminal diazo groups, e.g., the natural products Dynemicin, Kinamycin C, and Prekinamycin, as disclosed at http://www.indiana.edu/˜zalegrp/overview_acs.html. Others include, e.g., UVADEX and Methoxypsoralen (8-MOP) that are reported efficacious in extracorporeal photochemotherapy (ECP), as well as TER286 [g-glutamyl-a-amino-b(2-ethyl-N,N,N′,N′-tetrakis (2-chloroethyl)phos-phorodiamidate)-sulfonyl)-propionyl-(R)-(−)phenylglycine] (a nitrogen mustard prodrug) and 35S]-azidophenacyl-glutathione ([35S]APA-SG), chlorins, porphyrins, and phthalocyanines. See, e.g., http://www.ibmtindy.com/faq/photopheresis.htm; http://www.fccc.edu/research/reports/current/tew.reportframe.html http://www.info.med.yale.edu/dermatology/clinical/photo.html; http://search.msn.com/results.asp?FORM=sCPN&RS=CHECKED&un=doc&v=1&q=photoactivatable%20drug; Qualls et al., “Synergistic Phototoxicity of Chloroaluminum Phthalocyanine Tetrasulfonate Delivered via Acid-Labile Diplasmenylcholine-Folate Liposomes”, International Journal of Cancer 2001, 93, 384-392. Each of the preceding is herein incorporated by reference.

The label used may directly produce a signal. Alternatively, the label may indirectly produce a signal and therefore require additional reagents and/or physical stimulation, e.g., bombardment with electromagnetic energy or the addition of a chemical substrate or co-factor. In the instance of fluorescers, for example, these are able to absorb ultraviolet and visible light where the light absorption transfers energy to these molecules and elevates them to an excited energy state, which absorbed energy is then dissipated by emission of light at a second wavelength. By contrast, labels that directly produce a signal include, e.g., radioactive isotopes and dyes.

Examples of labels that need other reagent components to produce a Signal include, e.g., substrates and coenzymes (for enzyme labels), substances that react with enzymic products, catalysts, activators, cofactors, inhibitors, scavengers, metal ions, and a specific binding substance required for binding of signal generating substances. Additional discussion of suitable labeling systems can be found, e.g., in Ullman, et al., U.S. Pat. No. 5,185,243, columns 11-13, the disclosure of which is herein incorporated by reference.

The assay methods of the invention may be conveniently performed on a solid support, such as in multi-well plates for an ELISA or on any solid support for high density or chip array analysis, for example, in an ELISA type format, the ligand or receptor molecule is adsorbed to a solid support such as the wells of a 96-well plate. The corresponding complement (receptor or ligand, whichever the case may be), is/are added to the wells and incubated. Alternatively, the complexes may first be formed and then adhered to the solid support, to be competed with later by another ligand or compound of interest. In yet another embodiment, multiple ligands, at least one of which is known and labeled, are mixed together in the presence of an HSP90 and, by whichever of a broad variety of means known in the art, noncomplexed and nonadhered species (in solid support embodiments) are removed. The amount of label is then assessed using a detection device. Removal of noncomplexed and nonadhered species can be done by wash steps, and in some embodiments, centrifugation. Unbound ligand/receptor is washed away and the presence of labeled complex is then detected. Many variations are possible.

Detection hardware devices that can be used in connection with various embodiments of the present invention are well known in the art and include but are not limited to, e.g., densitometers, mass spectrometers, fluorometers, scintillation counters, spectrophotometers, nmr devices, positron emission tomography (PET) devices, luminometers, cameras, and other imaging or detection devices. These devices and their use are all well known in the art. For example, PET is described in Mandelkern et al., Positron emission tomography in cancer research and treatment. Technologies in Cancer Research and Treatment (2002) 1(6):423-39, and Zimny et al., Positron emission tomography scanning in gynecologic and breast cancers, Current Opinion in Obstetrics and Gynecology. (2003) 15(1):69-75.

Assays to Determine HSP90 Binding and Downstream Effect

In addition to the innovations described herein, a variety of in vitro and in vivo assays are available to test the effect of the compounds of the invention on HSP90. HSP90 competitive binding assays and functional assays can be performed as known in the art substituting in the compounds of the invention. Chiosis et al., Chemistry & Biology 8:289-299 (2001) describe some of the known ways in which this can be done. For example, competition binding assays using, e.g., geldanamycin or 17-AAG as a competitive binding inhibitor of HSP90 can be used to determine relative HSP90 affinity of the compounds of the invention by immobilizing the compound of interest or other competitive inhibitor on a gel or solid matrix, preincubating HSP90 with the other inhibitor, passing the preincubated mix over the gel or matrix, and then measuring the amount of HSP90 that sticks or does not stick to the gel or matrix.

Downstream effects can also be evaluated based on the known effect of HSP90 inhibition on function and stability of various steroid receptors and signaling proteins including, e.g., Raf1 and Her2. Compounds of the present invention induce dose-dependent degradation of these molecules, which can be measured using standard techniques. Inhibition of HSP90 also results in up-regulation of HSP90 and related chaperone proteins that can similarly be measured. Antiproliferative activity on various cancer cell lines can also be measured, as can morphological and functional differentiation related to HSP90 inhibition.

Many different types of methods are known in the art for determining protein concentrations and measuring or predicting the level of proteins within cells and in fluid samples. Indirect techniques include nucleic acid hybridization and amplification using, e.g., polymerase chain reaction (PCR). These techniques are known to the person of skill and are discussed, e.g., in Sambrook, Fritsch & Maniatis, Molecular Cloning: A Laboratory Manual, Second Edition (1989) Cold Spring Harbor Laboratory Press, Cold Spring Harbor, N.Y., Ausubel, et al., Current Protocols in Molecular Biology, John Wiley & Sons, NY, 1994, and, as specifically applied to the quantification, detection, and relative activity of Her-2/neu in patient samples, e.g., in U.S. Pat. Nos. 4,699,877, 4,918,162, 4,968,603; and 5,846,749. A brief discussion of two generic techniques that can be used follows.

The determination of whether cells over-express or contain elevated levels of HER-2 can be determined using well known antibody techniques such as immunoblotting, radioimmunoassays, western blotting, iminunoprecipitation, enzyme-linked immunosorbant assays (ELISA), and derivative techniques that make use of antibodies directed against HER-2. As an example, HER-2 expression in breast cancer cells can be determined with the use of an immunohistochemical assay, such as the Dako Hercep™ test (Dako Corp., Carpinteria, Calif.). The Hercep™ test is an antibody staining assay designed to detect HER-2 over-expression in tumor tissue specimens. This particular assay grades HER-2 expression into four levels: 0, 1, 2, and 3, with level 3 representing the highest level of HER-2 expression. Accurate quantitation can be enhanced by employing an Automated Cellular Imaging System (ACIS) as described, e.g., by Press, M, et al., 2000, Modern Pathology, 13:225A.

Antibodies, polyclonal or monoclonal, can be purchased from a variety of commercial suppliers, or may be manufactured using well-known methods, e.g., as described in Harlow et al, Antibodies: A Laboratory Manual, 2nd Ed; Cold Spring Harbor Laboratory Press, Cold Spring Harbor, N.Y. (1988).

HER-2 over-expression can also be determined at the nucleic acid level since there is a reported high correlation between over-expression of the HER-2 protein and amplification of the gene that codes for it. One way to test this is by using RT-PCR. The genomic and cDNA sequences for HER-2 are known. Specific DNA primers can be generated using standard, well-known techniques, and can then be used to amplify template already present in the cell. An example of this is described in Kurokawa, H. et al., Cancer Res. 60: 5887-5894 (2000). PCR can be standardized such that quantitative differences are observed as between normal and abnormal cells, e.g., cancerous and noncancerous cells. Well known methods employing, e.g., densitometry, can be used to quantitate and/or compare nucleic acid levels amplified using PCR.

Similarly, fluorescent in situ hybridization (FISH) assays and other assays can be used, e.g., Northern and/or Southern blotting. These rely on nucleic acid hybridization between the HER-2 gene or mRNA and a corresponding nucleic acid probe that can be designed in the same or a similar way as for PCR primers, above. See, e.g., Mitchell, M S, and Press, M F, 1999, Semin. Oncol., Suppl. 12:108-16. For FISH, this nucleic acid probe can be conjugated to a fluorescent molecule, e.g., fluorescein and/or rhodamine, that preferably does not interfere with hybridization, and which fluorescence can later be measured following hybridization. See, e.g., Kurokawa, H et al., Cancer Res. 60: 5887-5894 (2000) (describing a specific nucleic acid probe having sequence 5′-FAM-NucleicAcid-TAMRA-p-3′ sequence). ACIS-based approaches as described above can be employed to make the assay more quantitative (de la Torre-Bueno, J, et al, 2000, Modem Pathology 13:221 A).

Immuno and nucleic acid detection can also be directed against proteins other than HSP90 and Her-2, which proteins are nevertheless affected in response to HSP90 inhibition.

Pharmaceutical Compositions, Dosaging and Modes of Administration

Compounds identified as promising using the assays of the invention can in turn be formulated into pharmaceutical compositions and then administered to subjects.

Those of ordinary skill in the art are familiar with formulation and administration techniques that can be employed with the compounds and methods of the invention, e.g., as discussed in Goodman and Gilman, The Pharmacological Basis of Therapeutics, current edition; Pergamon Press; and Remington Pharmaceutical Sciences (current edition.), Mack Publishing Co., Easton, Pa., USA.

The compounds utilized in the methods of the instant invention may be administered either alone or in combination with pharmaceutically acceptable carriers, excipients or diluents, in a pharmaceutical composition, according to standard pharmaceutical practice. The compounds can be administered orally or parenterally, are nevertheless affected in response to HSP90 including the intravenous, intramuscular, intraperitoneal, subcutaneous, rectal and topical routes of administration.

For example, the therapeutic or pharmaceutical compositions of the invention can be administered locally to the area in need of treatment This may be achieved by, for example, but not limited to, local infusion during surgery, topical application, e.g., cream, ointment, injection, catheter, or implant, said implant made, e.g., out of a porous, non-porous, or gelatinous material, including membranes, such as sialastic membranes, or fibers. The administration can also be by direct injection at the site (or former site) of a tumor or neoplastic or pre-neoplastic tissue.

Still further, the compounds or compositions of the invention can be delivered in a vesicle, e.g., a liposome (see, for example, Langer, 1990, Science, 249:1527-1533; Treat et al., 1989, Liposomes in the Therapy of Infectious Disease and Cancer, Lopez-Bernstein and Fidler (Eds.), Liss, N.Y., USA, pp. 353-365).

The compounds and pharmaceutical compositions used in the methods of the present invention can also be delivered in a controlled release system. In one embodiment, a pump may be used (see, Sefton, 1987, CRC Grit. Ref. Biomed. Eng. 14:201; Buchwald et al., 1980, Surgery, 88:507; Saudek et al., 1989, N. Eng. J. Med., 321:574). Additionally, a controlled release system can be placed in proximity of the therapeutic target (see, e.g., Goodson, 1984, Medical Applications of Controlled Release, Vol. 2, pp. 115-138).

The pharmaceutical compositions used in the methods of the instant invention can also contain the active ingredient in a form suitable for oral use, for example, as tablets, troches, lozenges, aqueous or oily suspensions, dispersible powders or granules, emulsions, hard or soft capsules, or syrups or elixirs. Compositions intended for oral use may be prepared according-to any method known to the art for the manufacture of pharmaceutical compositions, and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, such as microcrystalline cellulose; sodium crosscarmellose, corn starch, or alginic acid; binding agents, for example starch, gelatin, polyvinyl-pyrrolidone or acacia, and lubricating agents, for example, magnesium stearate, stearic acid or talc. The tablets may be un-coated or coated by known techniques to mask the taste of the drug or delay disintegration and absorption in the gastrointestinal tract and thereby provide a sustained action over a longer period. For example, a water soluble taste masking material such as hydroxypropylmethyl-cellulose or hydroxypropyl cellulose, or a time delay material such as ethyl cellulose, or cellulose acetate butyrate may be employed as appropriate.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethyleneglycol or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethyl cellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol aziliydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present. These compositions may be preserved by the addition of an anti-oxidant such as ascorbic acid.

The compounds and pharmaceutical compositions used in the methods of the instant invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening agents, flavoring agents, preservatives and antioxidants.

Syrups and elixirs may be formulated with sweetening agents, e.g., glycerol, propylene glycol: sorbitol or sucrose. Such formulations may also contain a demulcent, a preservative, flavoring and coloring agents and antioxidant.

The pharmaceutical compositions may be in the form of sterile injectable aqueous solutions. Acceptable vehicles and solvents that may include, e.g., water, Ringer's solution and isotonic sodium chloride solution.

The sterile injectable preparation may also be a sterile injectable oil-in-water microemulsion where the active ingredient is dissolved in the oily phase. For example, the active ingredient may be first dissolved in a mixture of soybean oil and lecithin. The oil solution is then introduced into a water and glycerol mixture and processed to form a micro emulsion.

The injectable solutions or micro emulsions may be introduced into a patient's blood-stream by local bolus injection. Alternatively, it may be advantageous to administer the solution or micro emulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

The pharmaceutical compositions may be in the form of a sterile injectable aqueous or oleagenous suspension for intramuscular and subcutaneous administration. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane dial. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The Compounds of the present invention used in the methods of the present invention may also be administered in the form of suppositories for rectal administration of the drug. These compositions can be prepared by mixing the inhibitors with a suitable non-irritating excipient which is solid at ordinary temperatures but liquid at the rectal temperature and will therefore melt in the rectum to release the drug. Such materials include cocoa butter, glycerinated gelatin, hydrogenated vegetable oils, mixtures of polyethylene glycols of various molecular weights and fatty acid esters of polyethylene glycol.

For topical use, creams, ointments, jellies, solutions or suspensions, etc., containing a compound or composition of the invention can be used. As used herein, topical application can include, e.g., mouth washes and gargles.

The compounds used in the methods of the present invention can be administered in intranasal form via topical use of suitable intranasal vehicles and delivery devices, or via trans dermal routes, using those forms of transdermal skin patches well known to those of ordinary skill in the art. To be administered in the form of a transdermal delivery system, the dosage administration will, of course, be continuous rather than intermittent throughout the dosage regimen.

The methods, compounds and compositions of the instant invention may also be used in conjunction with other well known therapeutic agents that are selected for their particular usefulness against the condition that is being treated. For example, the instant compounds may be useful in combination with known anti-cancer and cytotoxic agents. Further, the instant methods and compounds may also be useful in combination with other inhibitors of parts of the signaling pathway that links cell surface growth factor receptors to nuclear signals initiating cellular proliferation.

The methods of the present invention may also be useful with other agents that inhibit angiogenesis and thereby inhibit the growth and invasiveness of tumor cells, including, but not limited to VEGF receptor inhibitors, including ribozymes and antisense targeted to VEGF receDtors. angiostatin and endostatin.

Examples of antineoplastic agents that can be used in combination with the compounds and methods of the present invention include, in general, and as appropriate, alkylating agents, anti-metabolites; epidophyllotoxin; an antineoplastic enzyme; a topoisomerase inhibitor; procarbazine; mitoxantrone; platinum coordination complexes; biological response modifiers and growth inhibitors; hormonal/anti-hormonal therapeutic agents and haematopoietic growth factors. Exemplary classes of antineoplastics include theanthracycline family of drugs, the vinca drugs, the mitomycins, the bleomycins, the cytotoxic nucleosides, the epothilones, discodermolide, the pteridine family of drugs, diynenes and the podophyllotoxins. Particularly useful members of those classes include, for example, carminomycin, daunorubicin, aminopterin, methotrexate, methopterin, dichloromethotrexate, mitomycin C, porfiromycin, 5-fluorouracil, 6-mercaptopurine, gemcitabine, cytosine arabinoside, podophyllotoxin or podo-phyllotoxin derivatives such as etoposide, etoposide phosphate or teniposide, melphalan, vinblastine, vincristine, leurosidine, vindesine, leurosine, paclitaxel and the like. Other useful antineoplastic agents include estramustine, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-II, topotecan, ara-C, bicalutamide, flutamide, leuprolide, pyridobenzoindole derivatives, interferons and interleukins.

When a compound or composition of the invention is administered into a human subject, the daily dosage will normally be determined by the prescribing physician with the dosage generally varying according to the age, weight, and response of the individual patient, as well as the severity of the patient's symptoms.

In one exemplary application, a suitable amount of compound is administered to a mammal undergoing treatment for cancer, for example, breast cancer. Administration typically occurs in an amount of between about 0.01 mg/kg of body weight to about 100 mg/kg of body weight per day (administered in single or divided doses), more preferably at least about 0.1 mg/kg of body weight per day. A particular therapeutic dosage can include, e.g., from about 0.01 mg to about 1000 mg of compound, and preferably includes, e.g., from about 1 mg to about 1000 mg. The quantity of active compound in a unit of preparation may be varied or adjusted from about 0.1 mg to 1000 mg, preferably from about 1 mg to 300 mg, more preferably 10 mg to 200 mg, according to the particular application. The amount administered will vary depending on the particular IC50 value of the compound used and the judgment of the attending clinician taking into consideration factors such as health, weight, and age. In combinational applications in which the compound is not the sole active ingredient, it may be possible to administer lesser amounts of compound and still have therapeutic or prophylactic effect.

Preferably, the pharmaceutical preparation is in unit dosage form. In such form, the preparation is subdivided into unit doses containing appropriate quantities of the active component, e.g., an effective amount to achieve the desired purpose.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The amount and frequency of administration of the compounds and compositions of the present invention used in the methods of the present invention, and if applicable other chemotherapeutic agents and/or radiation therapy, will be regulated according to the judgment of the attending clinician (physician) considering such factors as age, condition and size of the patient as well as severity of the disease being treated.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the patient, and in view of the observed responses of the disease to the administered therapeutic agents.

Also, in general, the compounds of the invention need not be administered in the same pharmaceutical composition as a chemotherapeutic agent, and may, because of different physical and chemical characteristics, be administered by a different route. For example, the compounds/compositions may be administered orally to generate and maintain good blood levels thereof, while the chemotherapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed.

The particular choice of compound (and where appropriate, chemotherapeutic agent and/or radiation) will depend upon the diagnosis of the attending physicians and their judgment of the condition of the patient and the appropriate treatment protocol.

The compounds/compositions of the invention (and where appropriate chemotherapeutic agent and/or radiation) may be administered concurrently (e.g., simultaneously, essentially simultaneously or within the same treatment protocol) or sequentially, depending upon the nature of the proliferative disease, the condition of the patient, and the actual choice of chemotherapeutic agent and/or radiation to be administered in conjunction (i.e., within a single treatment protocol) with the compound/composition.

In combinational applications and uses, the compound/composition and the chemotherapeutic agent and/or radiation are not administered simultaneously or essentially simultaneously, then the initial order of administration of the compound/composition, and the chemotherapeutic agent and/or radiation, may not be important. Thus, the compounds/compositions of the invention may be administered first followed by the administration of the chemotherapeutic agent and/or radiation; or the chemotherapeutic agent and/or radiation may be administered first followed by the administration of the compounds/compositions of the invention. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient. For example, the chemotherapeutic agent and/or radiation may be administered first, especially if it is a cytotoxic agent, and then the treatment continued with the administration of the compounds/compositions of the invention followed, where determined advantageous, by the administration of the chemotherapeutic agent and/or radiation, and so on until the treatment protocol is complete.

Thus, in accordance with experience and knowledge, the practicing physician can modify each protocol for the administration of a compound/composition for treatment according to the individual patient's needs, as the treatment proceeds.

The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help evaluate treatment efficacy.

### EXAMPLES

The following examples are offered by way of illustration only and not by way of limitation. Examples 1-3 illustrate alternative methods of producing a biotinylated geldanamycin, of formula/compound 5. Example 4 illustrates that such a compound is useful in competitive binding assays with other HSP90 ligands, e.g., other ansamycins such as geldanamycin and 17-AAG. Example 5 illustrates how HSP90s taken from tumor cell lines more avidly bind known HSP90 ligands. Example 6 demonstrates the selective accumulation and persistence of CF237 (an ansamycin dimer) in mouse and human tumors. Example 7 illustrates prophetic examples of preparing radionuclide-functionalized or cytotoxin-functionalized geldanamycin derivatives.

### Example 1

### Synthesis of Biotinylated Ansamycins Useful for Competition Binding Studies with HSP90

This example follows the scheme: 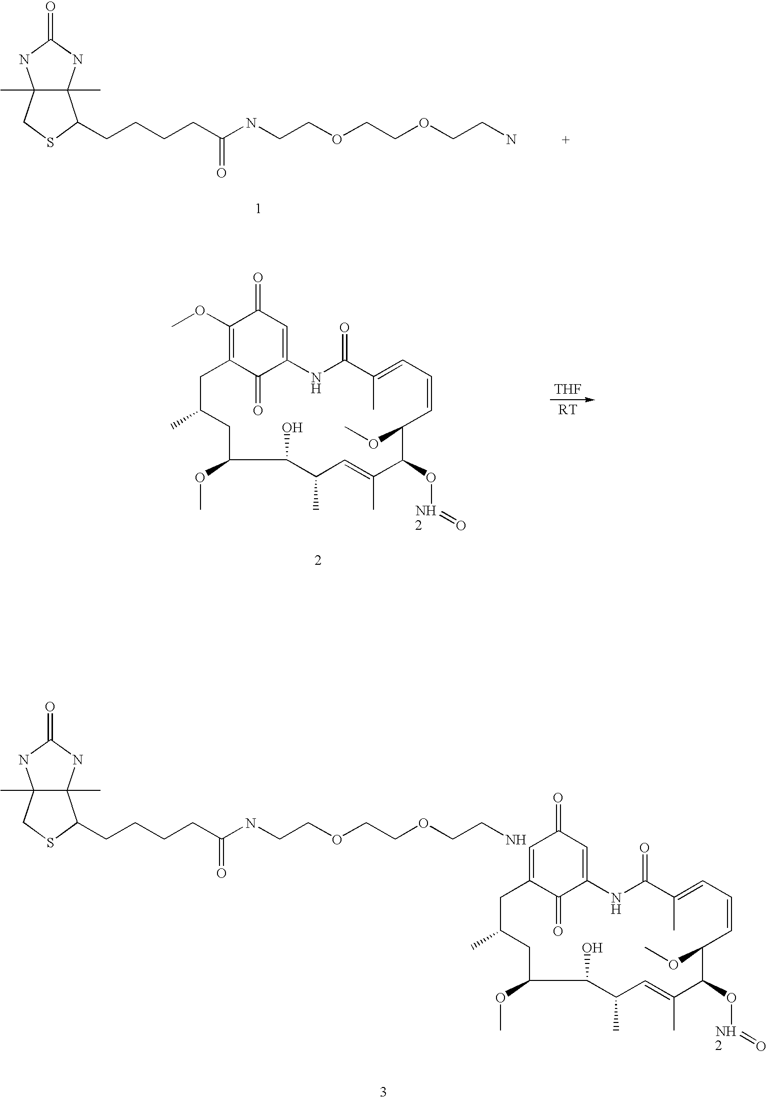
wherein the compound numbers are denoted beneath the corresponding structures, and wherein the details of the synthesis are as follows.

To 50 mg (0.134 mmol) of (+)-biotinyl-3,6-dioxaoctanediamine 1 in 3 mL of 15:1 THF—H20 was added 29.9 (0.053 mmol) of geldanamycin 2 at room temperature. The reaction was stirred overnight, quenched with water (50 mL) and extracted with 2×50 mL of EtOAc. The EtOAc extracts were combined, washed with 2×50 mL of H2O, 1×50 mL of brine, dried (MgSO4) and purified by silica gel flash chromatography to give 88 mg (0.095 mmol) in 71% yield of 3. MP 113-117° C.: MS 926 (M+Na).

### Example 2

### Synthesis of Biotinylated Ansamycins Useful for Competition Binding Studies with HSP90

This example follows the scheme: 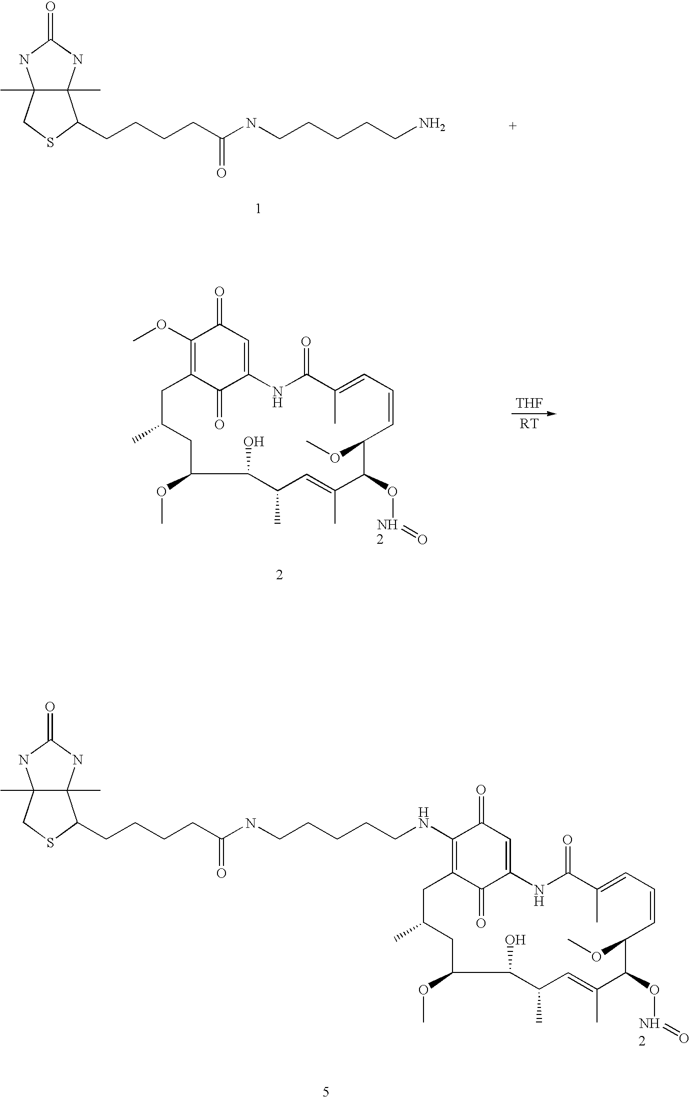
wherein the compound numbers are denoted beneath the corresponding structures, and wherein the details of the synthesis are as follows.

To 50 mg (0.152 mmol) of 5-(biotinamido)pentylamine 4 in 3 mL of 15:1 THF—H2O was added 28 mg (0.050 mmol) of geldanamycin 2 at room temperature. The reaction was stirred overnight, quenched with water (50 mL) and extracted with 2×50 mL of EtOAc. The EtOAc extracts were combined, washed with 2×50 mL of H2O, 1×50 mL of brine, dried (MgSO4) and purified by silica gel flash chromatography to give 100 mg (0.114 mmol) of 5 in 75% yield. MP 143-147° C. MS 880 (M+Na).

### Example 3

### Synthesis of Biotinylated Ansamycins Useful for Competition Binding Studies with HSP90

This example follows the scheme: 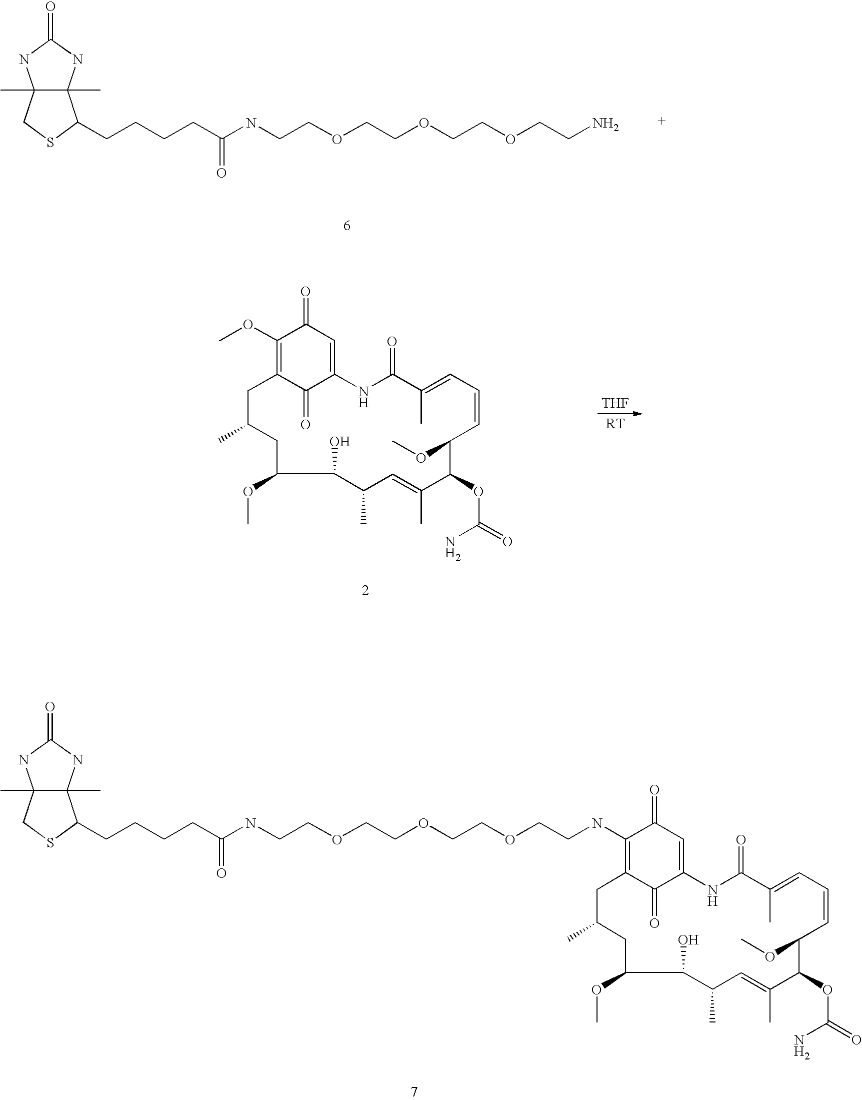
wherein the compound numbers are denoted beneath the corresponding structures and the details of the synthesis are as follows.

To 50 mg (0.119 mmol) of (+)-biotinyl-3,6,9-trioxaundecanediamine 6 in 3 mL of 15:1 THF—H20 was added 26.8 mg (0.048 mmol) of geldanamycin 2 at room temperature. The reaction was stirred overnight, quenched with water (50 mL) and extracted with 2×50 mL of EtOAc. The EtOAc extracts were combined, washed with 2×50 mL of H2O, 1×50 mL of brine, dried (MgSO4) and purified by silica gel flash chromatography to give 84 mg (0.087 mmol) of 7 in 73% yield. MP 103-104° C. MS 970 (M+Na).

### Example 4

### HSP90 Binding Assay Utilizing a Biotinylated Ansamycin

Purified native HSP90 protein was coated onto 96-well plates by incubating for 1 hr. at 37° C. Uncoated HSP90 was removed and the wells washed twice in 1× PBS (phosphate-buffered saline) buffer. Compound 5 (biotinylated geldanamycin) was then added to the wells, and the reaction was further incubated for 1 hr at 37° C. The wells were washed twice with 1× PBS, before the addition of 20 ug/ml streptavidin-phycoerythrin, and incubated for 1 hr at 37° C. The wells were again washed twice with 1× PBS. The fluorescence was then measured in a Gemini Spectrofluorometer (Molecular Devices Corp, Sunnyvale, Calif., USA) using an excitation of 485 nm and emission of 580 nm.

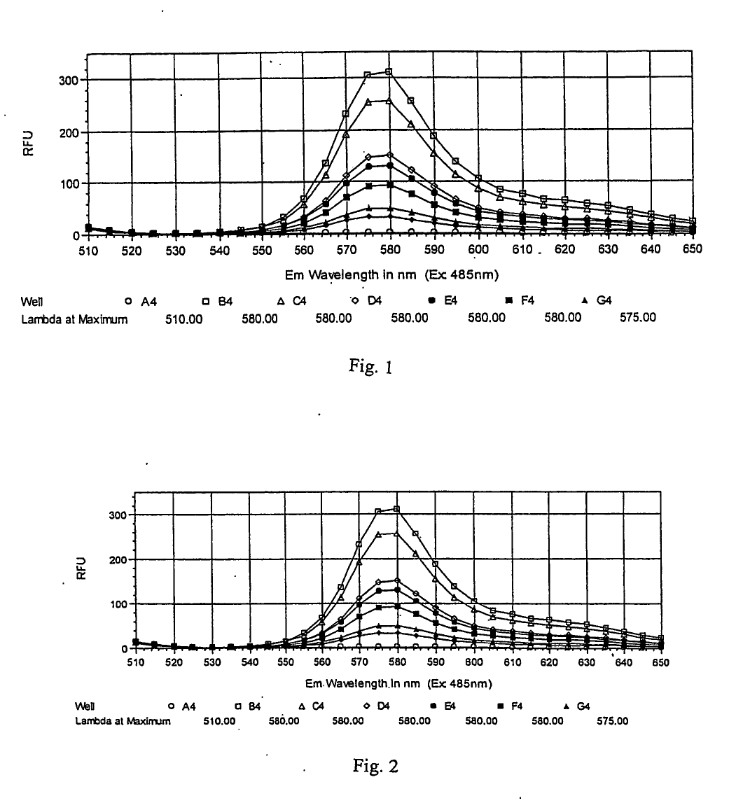

FIGS. 1-3 show assay embodiments employing different HSP90 ligands. Details of the assays are as follows:

FIG. 1. Competition of biotinylated-GM (compound 5) binding by free geldanamycin (GM). Native HSP90 that was coated onto 96-well plates was pre-incubated with increasing concentrations of 0 nM (B3), 100 nM(C3), 300 nM (D3), 1000 nM (E3), 3000 nM (F3), 10,000 nM (G3), and 100,000 nM (closed diamonds) of geldanamycin and then 5 was added. Binding of 5 was detected by measuring the fluorescence of streptavidin-phycoerythrin (excitation: 485 run; emission: 510-650 run). The background fluorescence without any HSP90 present (A3) was minimal. Increasing concentrations of GM inhibits the peak fluorescence at 580 run.

FIG. 2. Competition of biotinylated-GM (compound 5) binding by 17-allyl amino geldanamycin (17-AAG). Native Hsp90 that was coated onto 96-well plates was pre-incubated with increasing concentrations of 0 nM (B4), 100 nM (C4), 300 nM (D4), 1000 nM (E4), 3000 nM (F4), 10,000 nM (G4), and 100,000 nM (closed diamonds) of 17-AAG and then 5 was added. Binding of 5 was detected by measuring the fluorescence of streptavidin-phycoerythrin (excitation: 485 nm; emission: 510-650 nm). The background fluorescence without any HSP90 present (A4) was minimal. Increasing concentrations of 17-AAG inhibits—the peak fluorescence at 580 nm

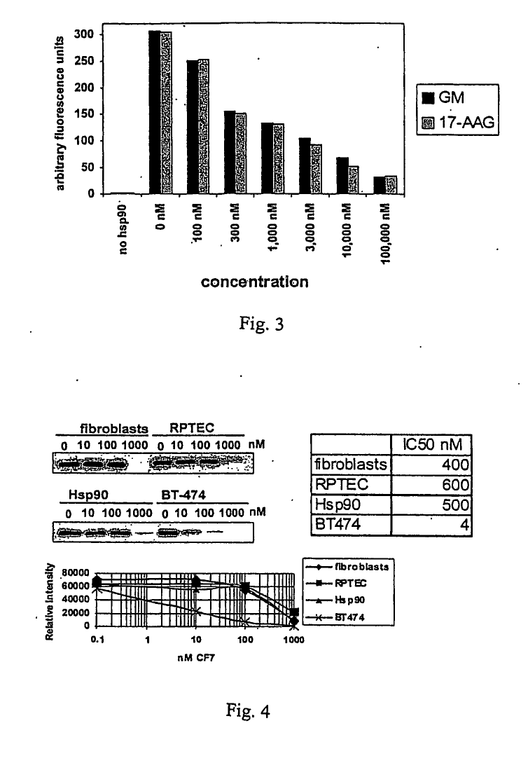

FIG. 3. Competition of biotinylated-GM (compound 5) binding by geldanamycin (GM) and 17-allyl amino geldanamycin (17-AAG). Native Hsp90 that was coated onto 96-well plates was pre-intubated, with increasing concentrations of 0-100,000 nM of either GM or 17-AAG and then 5 was added Binding of 5 was detected by measuring the fluorescence of streptavidin-phycoerythrin (excitation: 485 nm; emission: 580 nm). The background fluorescence without any HSP90 present was minimal. Increasing concentrations of GM or 17-AAG inhibits the peak fluorescence at 580nm.

### Example 5

### HSP90s Taken from Tumor Cell Lines More Avidly Bind HSP90 Ligands

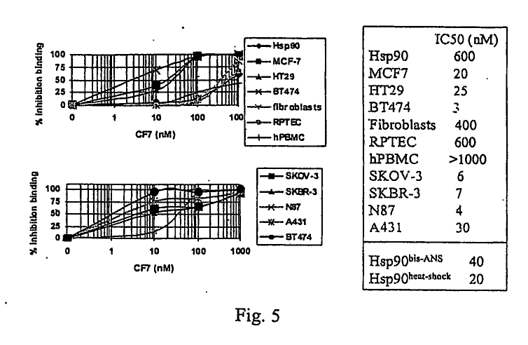

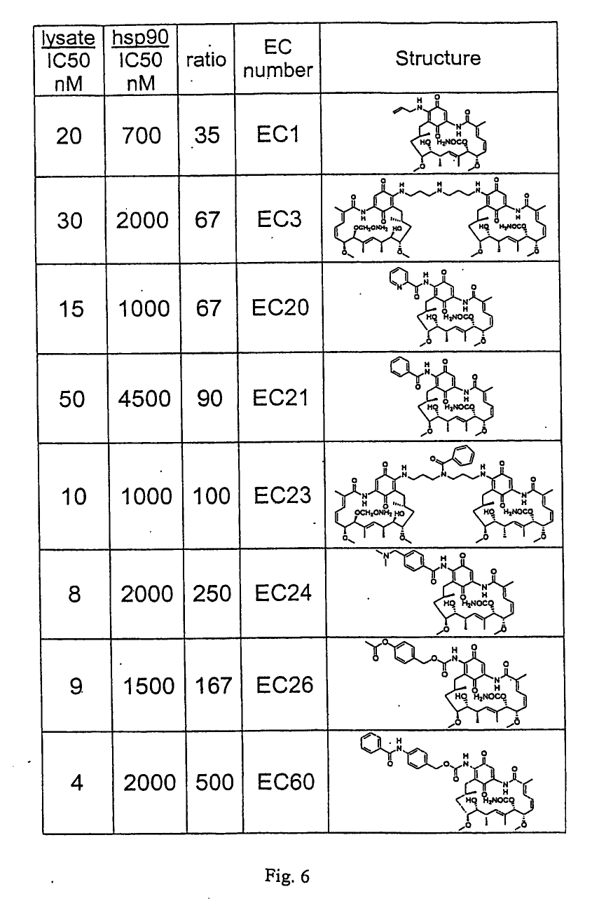

Purified native HSP90 protein (Stressgen) or cell lysates prepared in lysis buffer (20 mM Hepes, pH 7.3, 1 mM EDTA, 5 mM MgCl2, 100 mM KCl) were incubated in the absence or presence of CF7 (17-AAG) or test compound for 15 min at 4° C. Biotin-geldanamycin (biotin-GM) was then added to the mixture as discussed previously, and the reaction was further incubated by rotating for 1 hr at 4° C. BioMag™ streptavidin magnetic beads were then added to the mixture, and the reaction was incubated by rotating for another 1 hr at 4° C. Tubes were placed on a magnetic rack, and the unbound supernatant removed. The magnetic beads were washed three times in lysis buffer, and the washes discarded. SDS-PAGE sample buffer was added to the beads and boiled for 5 min at 95° C. Samples were analyzed on 10% SDS protein gels (Novex), and then Western blots using anti-HSP90 monoclonal antibody (Stressgen SPA-830). The bands in the Western Blots were quantitated using the Bio-rad Fluor-S Imager, and the % inhibition of binding of CF7 or test compound calculated. The IC50 reported is the concentration of the compound needed to cause half-maximal inhibition of binding. For experiments that utilized shocked HSP90, the purified HSP90 native protein was incubated for 15 min at 90° C. For experiments that utilized bis-ANS treated HSP90, the purified HSP90 protein was incubated with bis-ANS (Molecular Probes) for 30 min at 37° C. The results are shown in FIGS. 4-6.

### Example 6

### Pharmacokinetic Demonstration of Selective Accumulation and Persistence of CF237 in Human Tumors Relative to Normal Human Cells and Normal Mouse Tissues

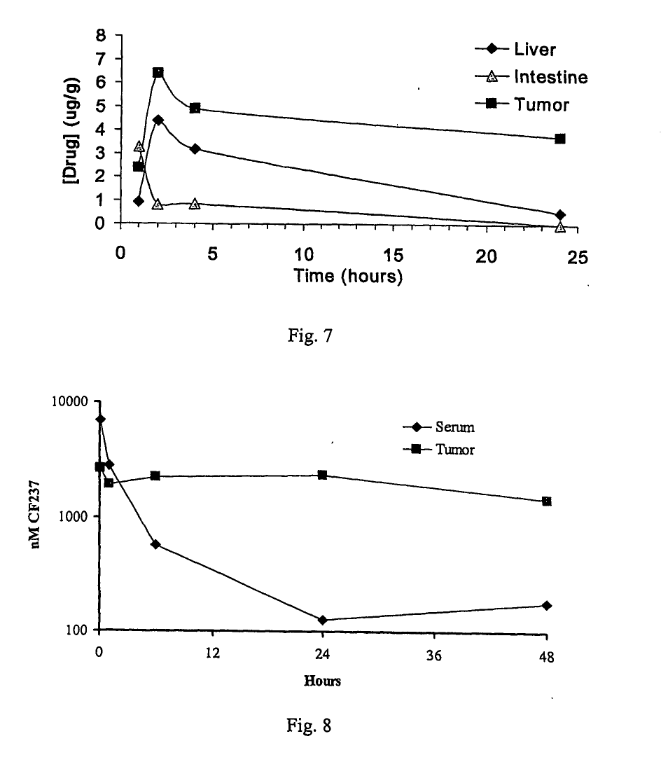

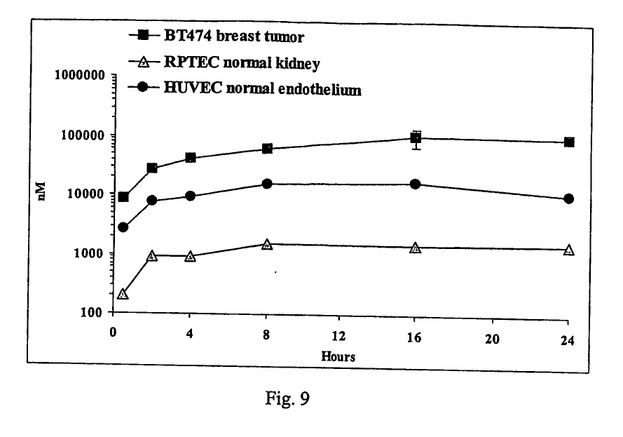

FIGS. 7 and 8 respectively show the pharmacokinetic profile of CF237 in human tumor xenografts relative to liver/intestine and serum of the host animals FIG. 9 shows the in vitro cellular drug uptake kinetics of CF237 in a human breast tumor cell line and in normal human epithelial and endothelial cells.

The preparation of CF237 is described in commonly owned application PCT/US03/0483, as is the preparation of many other ansamycins, including many geldanamycin derivatives that can be used in connection with various aspects and embodiments of the present invention.

Tumor tissue was a human tumor cell line, MDA435, obtained from the American Type Culture Collection (ATCC), Rockville, Md., USA. MDA-MB-435 is a human breast tumor line isolated in 1976 by R. Cailleau, et al. from the pleural effusion of a 31 year old female with metastatic, ductal adenocarcinoma of the breast. Cailleau R, et al. Long-term human breast carcinoma cell lines of metastatic origin: preliminary characterization, In Vitro 14: 911-915, 1978; PubMed: 730202. Normal human tissues were purchased from Clonetics, the BT474 tumor cells from ATCC.

Briefly, drug was administered in a single dose of 30 mg/kg body weight via the intraperitoneal route in a formulation comprising 6 parts PEG400, 1 part ethanol and 3 parts Tween 80 (“PET formulation”). Test animals (3/group) were sacrificed at the indicated times after dosing (as indicated in FIGS. 7 and 8). Tumors were excised, minced into 1 mm³ fragments and frozen at −80° C. in cryovials until ready for use, at which time they were thawed and homogenized in acetonitrile, and the solvent then evaporated off under nitrogen. Extractions were then performed on the homogenate using ethyl acetate/water. Blood samples were taken at the times indicated in FIG. 8 by serial bleed from the retro-orbital sinus or the tail vein. Blood was collected into tubes, allowed to clot at room temperature for one hour and serum was collected as the supernatant after centrifugation at 300×g for 5 minutes. The usual volume of serum available for analysis from the pharmacokinetics studies is 15 μL, although smaller volumes can be used. 485 μL acetonitrile and 15 μL serum sample (from each time point) are added to a 1.5 mL siliconized microcentrifuge tube and the mixture vortexed briefly at high speed. The tubes are then centrifuged at 21,000×g for 10 minutes, after which the organic layer is transferred to a glass test tube. The tubes containing the organic phase are then placed in a Turbovap set at 40° C. for 5-10 min. and the acetonitrile blown off using 25PSI nitrogen. Once the tubes are totally dry, the sample is reconstituted with 150 μL of Mobile Phase (30% Mobile Phase B Acetonitrile w/1% Acetic Acid and 0.5% TEA/70% Mobile Phase A H2O w/1% Acetic Acid and 0.5% TEA for ansamycins, 5% Mobile Phase B Acetonitrile with 0.05% TFA/95% Mobile Phase B H2O with 0.1% TFA). The tubes ar well mixed and the contents then transferred to HPLC vials or 96-well microplates with a micro-insert. HPLC is then performed on the samples at ˜160 Bar using an Agilent 1100 series device with a Zorbax 300SB—C₁₈ column (5uM particle size; 150×4.6mm), UV photodiode array detector, and the following phases of solvent (30% Mobile Phase B Acetonitrile w/1% Acetic Acid and 0.5% TEA/70% Mobile Phase A H2O w/1% Acetic Acid and 0.5% TEA for ansamycins, 5% Mobile Phase B Acetonitrile with 0.05% TFA/95% Mobile Phase B H2O with 0.1% TFA). The analysis takes approximately 15-25 minutes per sample, based on method. As controls, spiked serum standards using pooled Balb/C or Nu/Nu mouse serum containing 10, 25, 50, 500 and 5000 ng/mL of the compounds of interest were run for each analytical run per the following table.

| | | 1 mg/mL | |
|---|---|---|---|
| | | Standard | |
| | | (Compound | |
| Standard | Mouse | dissolved | |
| Concentration | Serum | in Mobile | Dilution |
| (ng/mL) | (μL) | Phase) | Scheme |
| 5000 | 497.5 | 2.5 | NA |
| 500 | | | 100 μL Mouse Serum + |
| | | | 10 μL 5000 ng/mL |
| | | | Standard |
| 50 | | | 200 μL Mouse Serum + |
| | | | 20 μL 500 ng/mL |
| | | | Standard |
| 25 | | | 50 μl Mouse Serum + |
| | | | 50 μl 50 ng/mL Standard |
| 10 | | | 100 μl Mouse Serum + |
| | | | 20 μl 50 ng/mL Standard |

Resulting data is expressed as ng/ml or nM for serum pharmacokinetics (PK) and as ng drug/g tissue for tumor PK

FIG. 9. 18× T75 of BT474 (Breast carcinoma) cells were compared to 9× T75 of RPTEC and HUVEC (normal cells). CF237 was added to flasks of these cells (80-90% confluent) at indicated times to a final concentration of 1 uM in 20 ml volumes. Incubations were at 37° C. Cells were harvested using 5 ml trypsin/flask at the times indicated in FIG. 9 (i.e. 0.5, 2, 4, 8, 16 and 24 hours) neutralized with 5 ml medium, spun down, resuspended in 1 ml PBS buffer, spun down again in an Eppendorf centrifuge for 1 min @13000 rpm, the supernatant aspirated and the product stored at −20° C. for later analysis. Analysis was made by suspending the pellets in 1 mL of lysing buffer on ice for 30 min, after which centrifugation was performed @14000 RPM for 15 min @4° C., and the product suspended in 3 mL of ethylated. Extracts were evaporated @20 psi for 15 min @30° C., reconstituted in MPA (25 mM Tris-HCl pH 7.0 40% ACN) and assayed by HPLC as described above. The results are displayed in the following table and in FIG. 9:

| | CF-237 | | | CF-237 | CF-237 | |
|---|---|---|---|---|---|---|
| Time | BT 474- | CF-237 | CF-237 | BT | BT | BT474- |
| Hour | mean | RPTEC | HUVEC | 474-01 | 474-02 | SD |
| 0.5 | 8570 | 203 | 2603 | 8838 | 8302 | 379.07 |
| 2 | 26500 | 894 | 7470 | 26759 | 26242 | 365.37 |
| 4 | 41647 | 905 | 9100 | 42530 | 40764 | 1248.61 |
| 8 | 61970 | 1520 | 15483 | 57883 | 66056 | 5779.42 |
| 16 | 101902 | 1450 | 16706 | 77287 | 126517 | 34810.46 |
| 24 | 97677 | 1485 | 11026 | 94726 | 100628 | 4173.41 |

### Example 7

### Prophetic Examples of Methods for Preparing Radionuclide-Functionalized or Cytotoxin-Functionalized Geldanamycin Derivatives

Methods for synthesizing nuclides and intermediates are described in Boehm et al., J. Org. Chem., 1986, 51, 5447-5450; Eng et al., Bull. Chim. Soc. Belges, 1986, 95, 895-914; Prestwich et al., Tetrahedron 1984, 40, 529-537; Hanson et al., Int. J. Appli. Radiat. Isot., 1984, 35, 810-812; Corey et al., J. Am. Chem. Soc., 1974, 96, 5581-5583; and Bottaro et al., J. Org. Chem., 1967, 174, 424-427, Synthesis and Applications of Isotopically Labelled Compounds, Volume 7 Ulrich Pleiss (Editor), Rolf Voges (Editor), ISBN: 0-47149501-8, January 2001; Jalilian et al. J Pharm Pharmaceut Sci. 2000, 3, 114-124. Two additional citations for preparing radioactive iodine compounds include John et al., An improved synthesis of [125I]N-(diethylaminoethyl)-4-iodobenzamide: a potential ligand for imaging malignant melanoma, Nuclear Medicine and Biology (1993), 20(1), 75-9. CODEN: NMBIEO ISSN:0883-2897. CAN 118:212605 AN 1993:212605 CAPLUS, and Chumpradit, S.; Kung, M. P.; Billings, J.; Mach, R.; Kung, H. F. Fluorinated and iodinated dopamine agents: D2 imaging agents for PET and SPECT. Journal of Medicinal Chemistry (1993), 36(2), 221-8. CODEN: JMCMAR ISSN:0022-2623. CAN 118:254670 AN 1993:254670 CAPLUS. Based on the foregoing, the following prophetic synthetic schemes and products are hypothesized to be produced and have useful effect according to various aspects and embodiments of the invention. 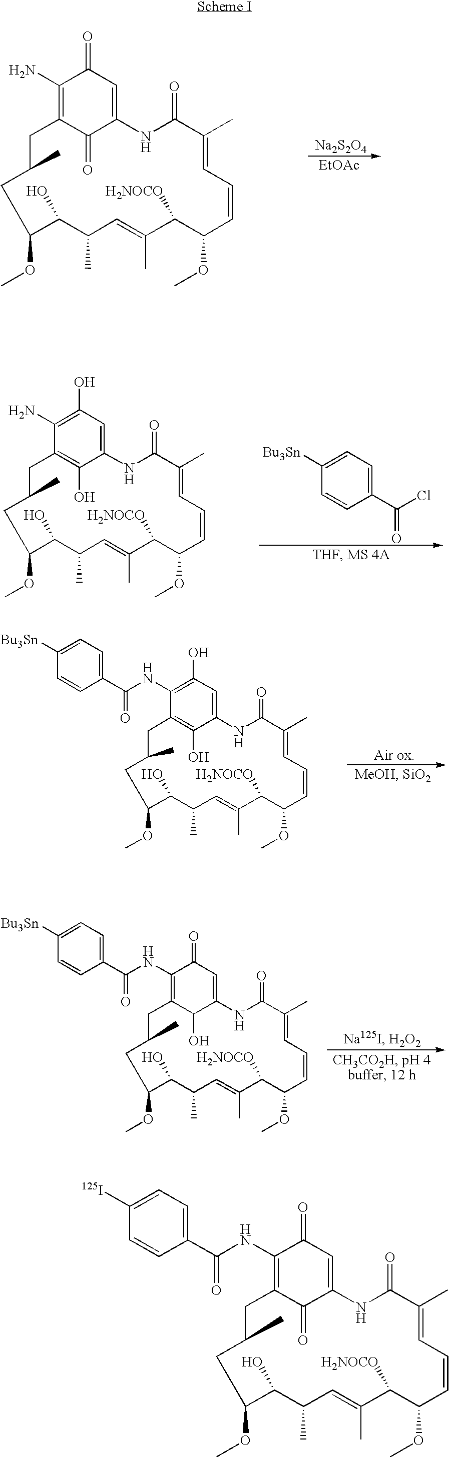 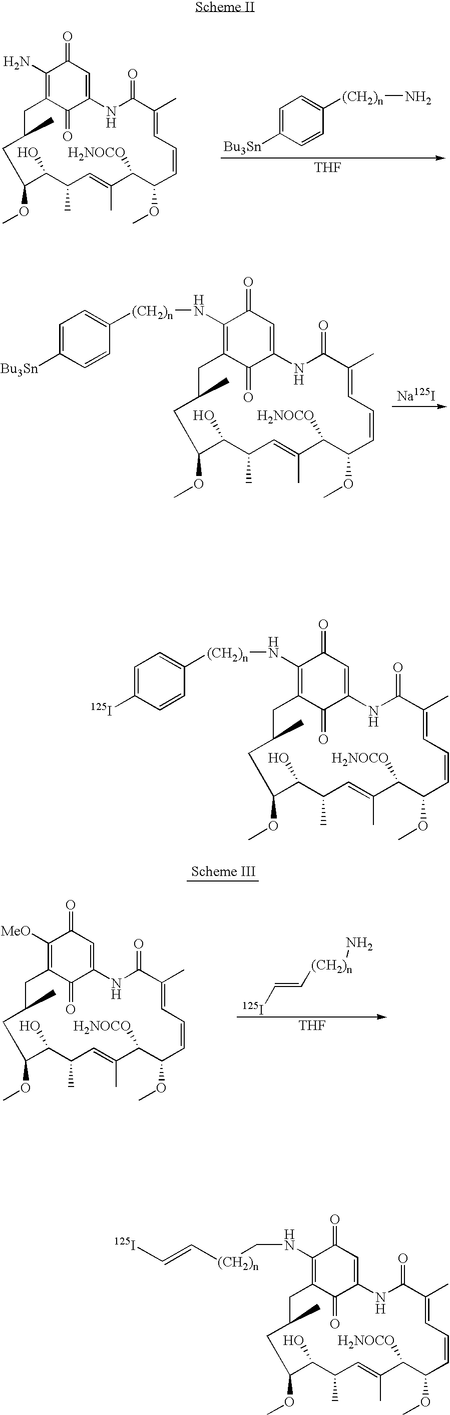 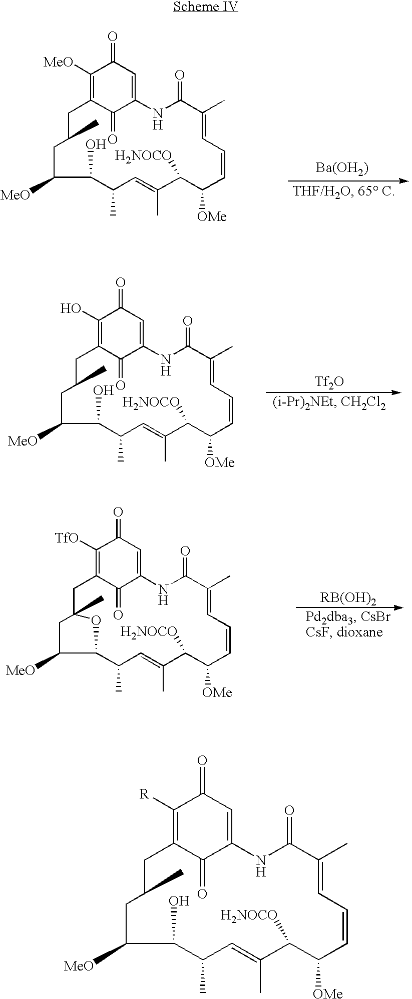 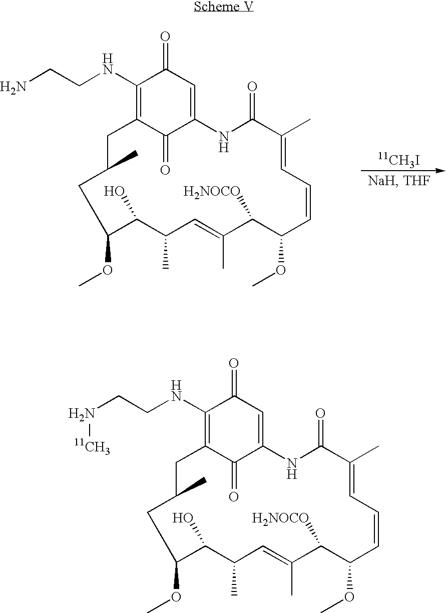 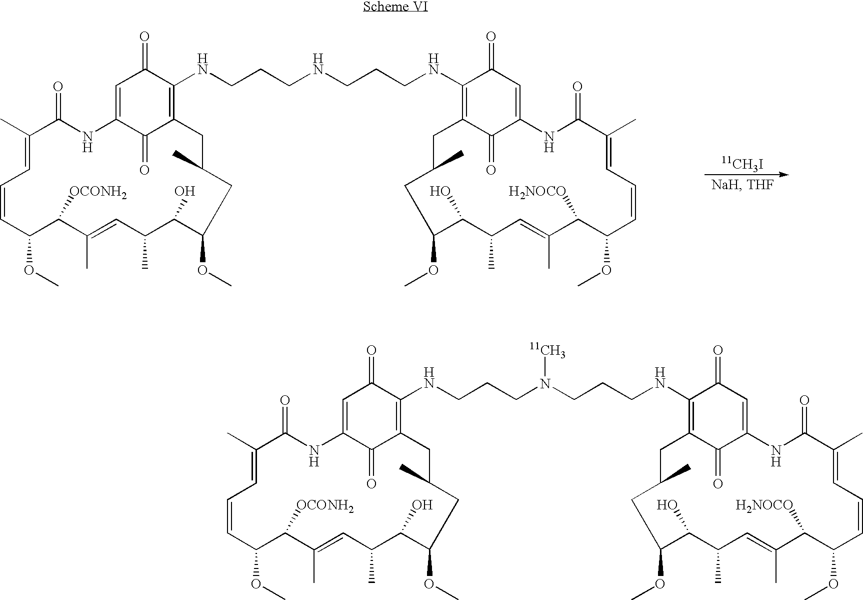 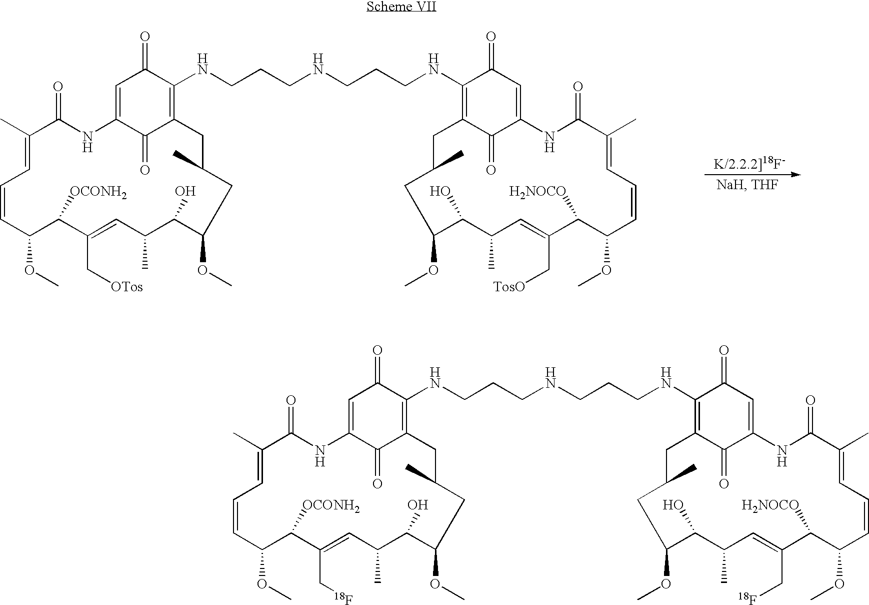
([K/2.2.2]¹⁸F:⁻ is an aminopolyether potassium complex [Kryotofix 2.2.2]¹⁸F⁻)

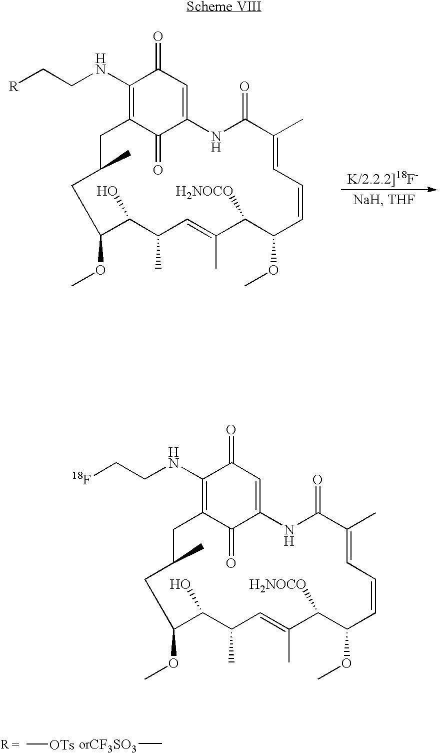

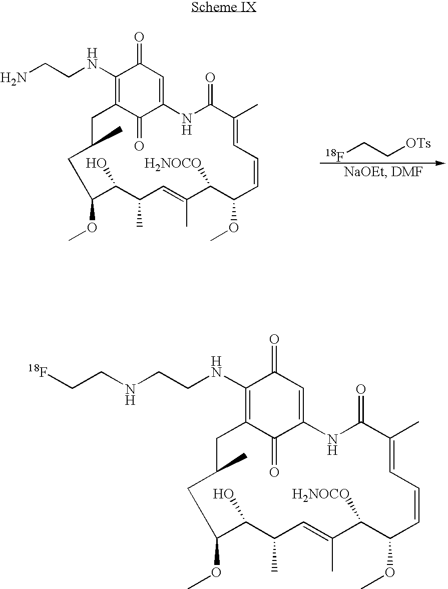

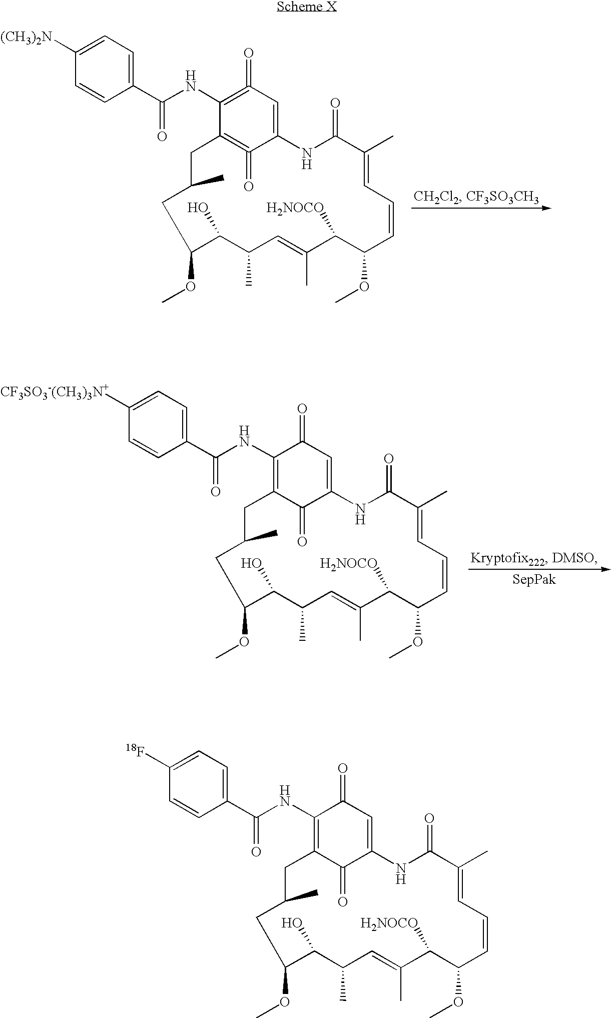

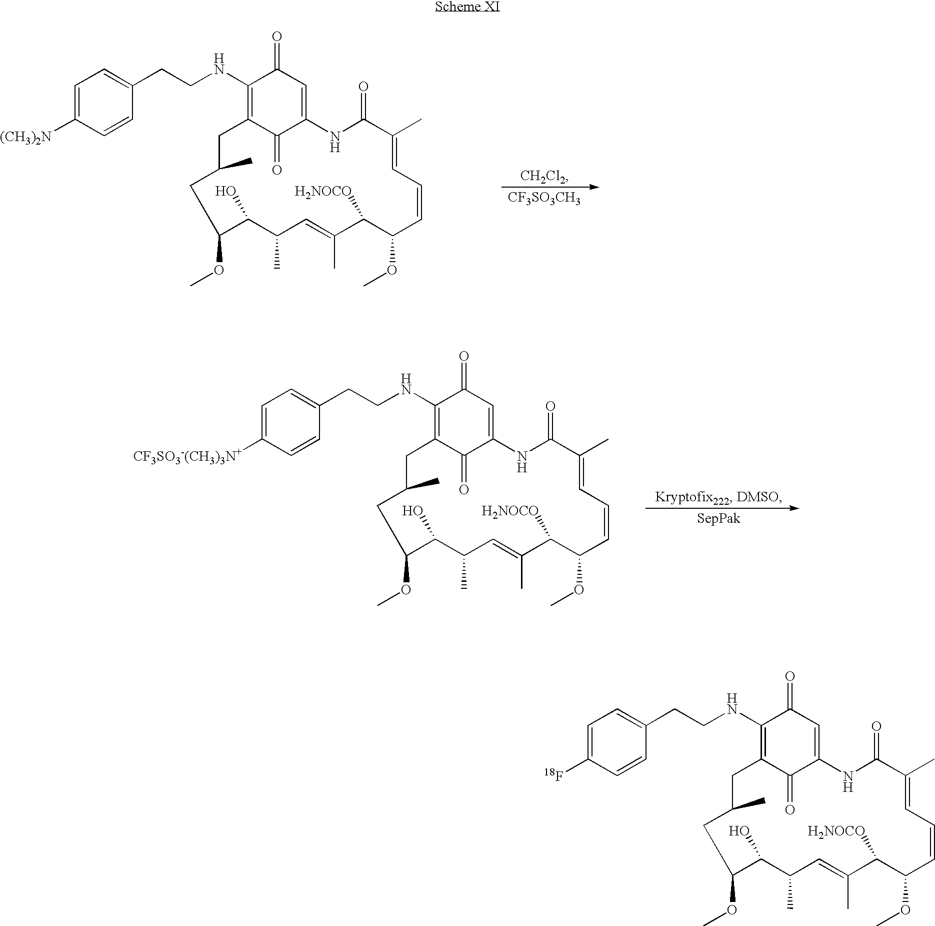

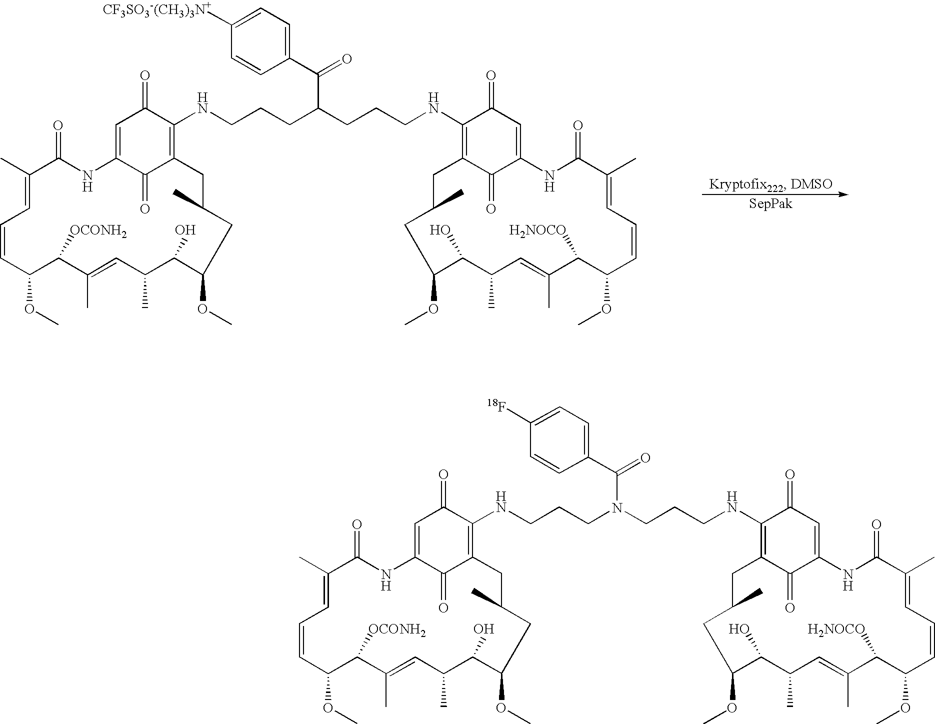

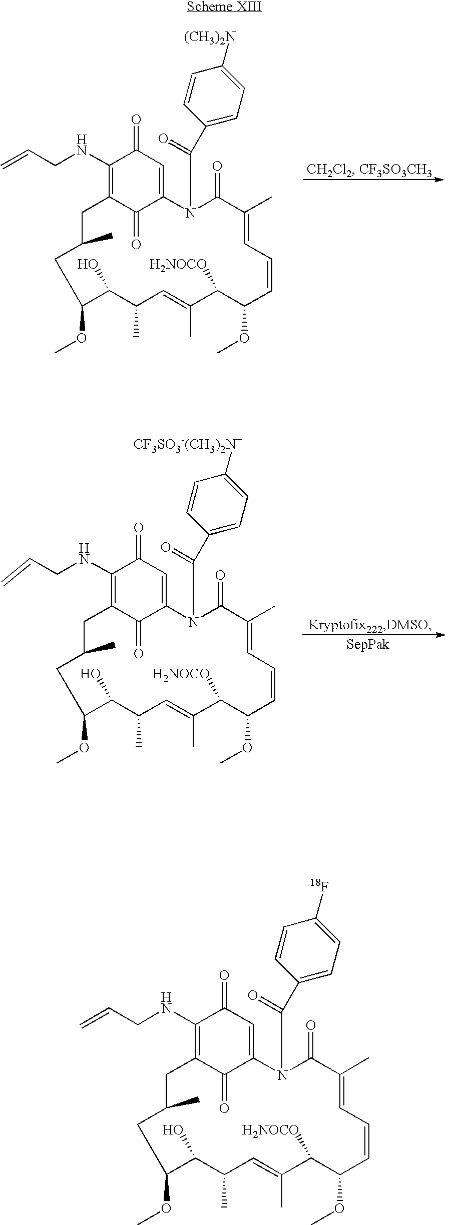

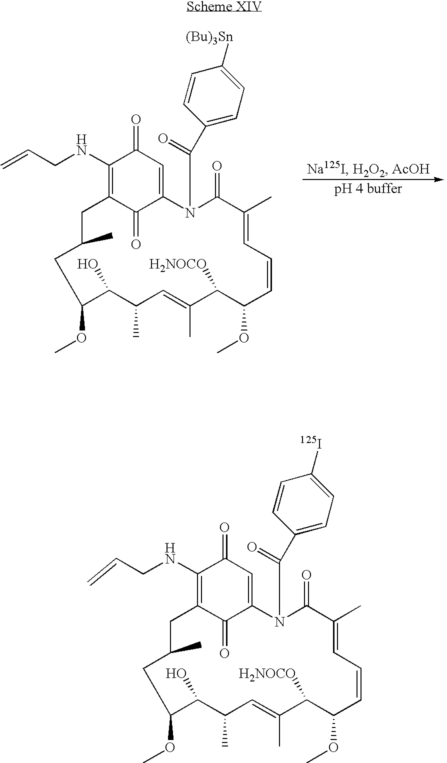

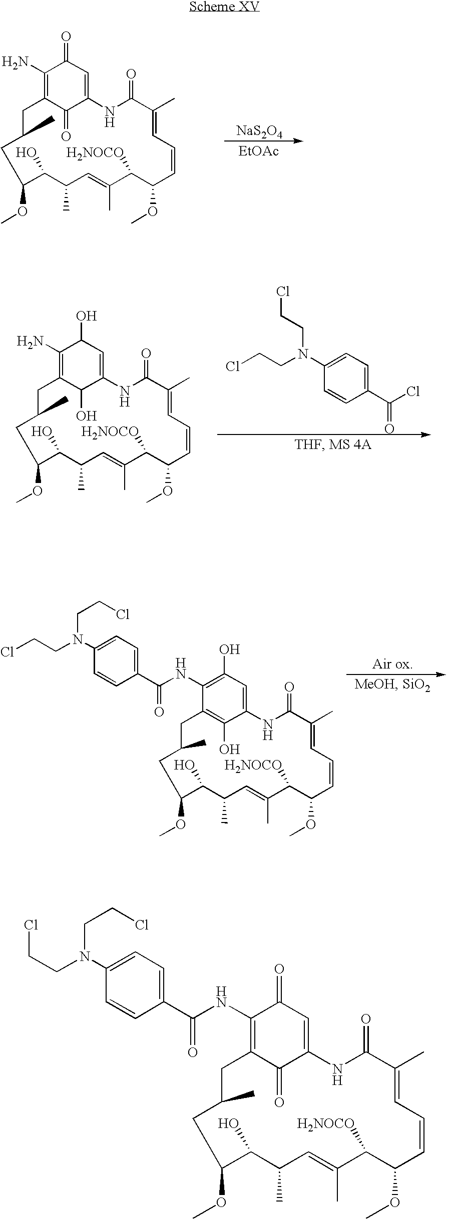

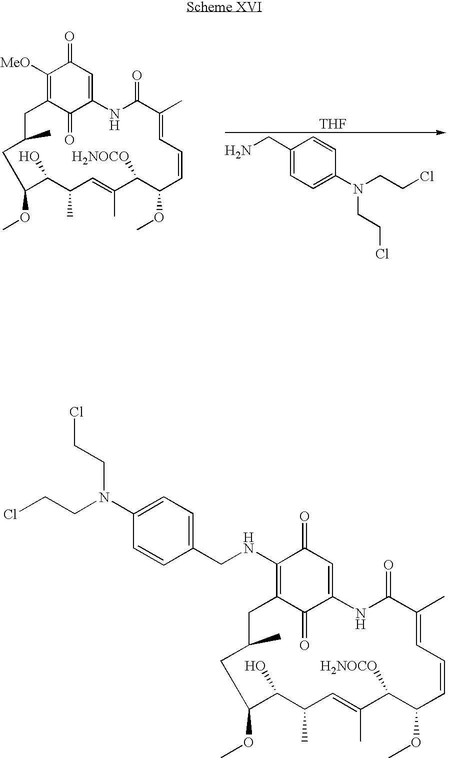

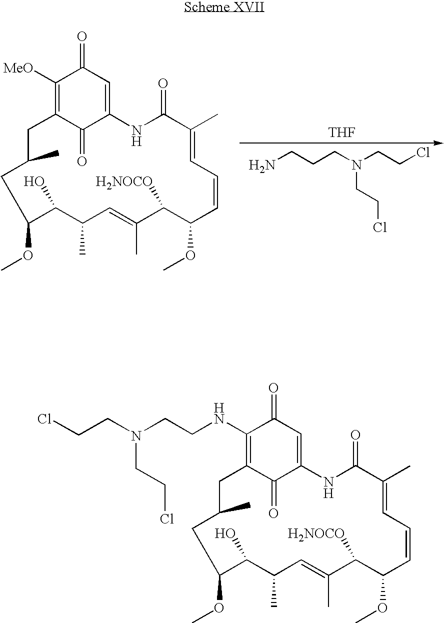

The foregoing examples are not limiting and are merely illustrative of various aspects and embodiments of the present invention. All documents cited are indicative of the levels of skill in the art to which the invention pertains. The reagents used, other than those novel reagents of the invention, are commercially available and/or readily synthesized or acquired by one of ordinary skill in the art without undue effort. The disclosure of each document is incorporated by reference herein to the same extent as if each had been incorporated by reference in its entirety individually, although none of the documents is admitted to be prior art.

One skilled in the art will readily appreciate that the present invention is well adapted to carry out the objects and obtain the ends and advantages mentioned, as well as those inherent therein. The methods and compositions described illustrate preferred embodiments, are exemplary, and are not intended as limitations on the scope of the invention. Certain modifications and other uses will occur to those skilled in the art, and are encompassed within the spirit of the invention, as defined by the scope of the claims.

It will be readily apparent to one skilled in the art that varying substitutions and modifications may be made to the invention without departing from the scope and spirit of the invention. Thus, such additional embodiments are within the scope of the invention and the following claims.

The invention illustratively described herein suitably may be practiced in the absence of any element or elements, limitation or limitations which is not specifically disclosed herein. Thus, for example, while the terms “comprising”, “consisting essentially of” and “consisting of,” each carries a different meaning as a transition phrase, each such phrase may be used in lieu of the others to demonstrate a different aspect or embodiment of the invention. The terms and expressions which have been employed are used as terms of description and not of limitation, and there is no intention in the use of such terms and expressions of excluding any equivalents of the features shown and described, or portions thereof. It is recognized that various modifications are possible within the scope of the invention claimed. Thus, it should be understood that although the present invention has been specifically disclosed by preferred embodiments, optional features, modifications and variations of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group, and exclusions of individual members as appropriate.

Other embodiments are within the following claims.

We claim:

1. A composition comprising a compound, said compound comprising an HSP90 ligand selected from the group consisting of purines, ansamycins, radicicol, zearalanols, ATP analogs, indoles, chalcones, and benzimidazoles; said HSP90 ligand derivatized with a functional moiety selected from the group consisting of imaging agents, radioactive therapeutic agents, and cytotoxic agents.

2. The composition of claim 1 wherein said ligand is geldanamycin derivatized at one or more of the −8, −11, and −17 positions with said functional moiety.

3. The composition of claim 1 wherein said ligand is geldanamycin derivatized at position −17 with said functional moiety.

4. The composition of any of claims 1-3 wherein said ligand has formula A 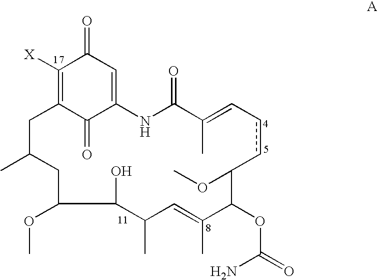
wherein X comprises said functional moiety, and wherein positions 4 and 5 are optionally both hydrated (4,5-dihydrogeldanamycin).

5. The composition of any-one of claims 1-3 wherein said functional moiety comprises a radioisotope.

6. The composition of any one of claims 1-3 wherein said functional moiety comprises a radioisotope selected from the group consisting of Iodine¹²⁵, Iodine¹³¹, ²¹³Bi, Technitium^{99m}, Technitium⁹⁹, Indium¹¹¹, Rhenium¹⁸⁸, Gallium⁶⁷, Copper⁶⁷, Yttrium⁹⁰, and Astatine²¹¹.

7. The composition of claim 5 wherein said radioisotope is selected from the group consisting of ¹⁸F, ¹¹C, ¹³N, ¹²³I, ¹²⁴I, ¹²⁵I, ¹³¹I, and ¹⁵O.

8. The composition of claim 1 wherein said compound has a formula selected from among the following group of formulas

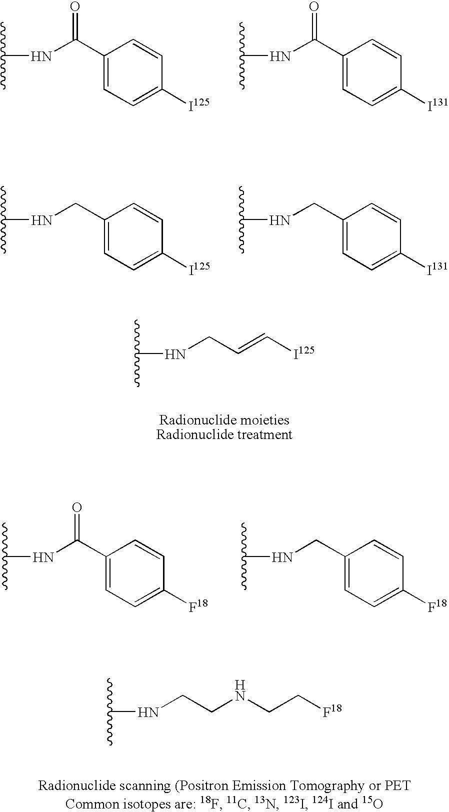

9. The composition of any one of claims 1-3 wherein said functional moiety comprises an imaging agent.

10. The composition of any one of claims 1-5 wherein said functional moiety comprises a radioactive therapeutic agent.

11. The composition of claim 1 wherein said functional moiety is selected from the group consisting of radioisotopes, antibodies, recombinant products, small molecules, antineoplastic agents, nitrogen mustard drugs (mustins), herceptin, taxol, taxanes and taxane derivatives, gleevec, alkylating agents, anti-metabilites; epidophyllotoxin; an antineoplastic enzyme; a topoisomerase inhibitor; procarbazine; mitoxantrone; platinum coordination complexes; biological response modifiers/growth inhibitors; hormonal/anti-hormonal therapeutic agents and haematopoietic growth factors, anthracycline drugs, vinca drugs, mitomycins, bleomycins, cytotoxic nucleosides, tepothilones, discodermolide, pteridine drugs, diynenes, podophyllotoxins, carminomycin, daunorubicin, aminopterin, methotrexate, methopterin, dichloromethotrexate, mitomycin C, porfiromycin, 5-fluorouracil, 6-mercaptopurine, gemcitabine, cytosine arabinoside, podophyllotoxin, podo-phyllotoxin derivatives, etoposide, etoposide phosphate or teniposide, melphalan, vinblastine, vincristine, leurosidine, vindesine, leurosine, paclitaxel, estramustine, carboplatin, cyclophosphamide, bleomycin, gemcitibine, ifosamide, melphalan, hexamethyl melamine, thiotepa, cytarabin, idatrexate, trimetrexate, dacarbazine, L-asparaginase, camptothecin, CPT-11, topotecan, ara-C, bicalutamide, flutamide, leuprolide, pyridobenzoindole derivatives, interferons and interleukins, and photoactivatable compounds.

12. The composition of claim 1 wherein said compound has a formula selected from the following group of formulas

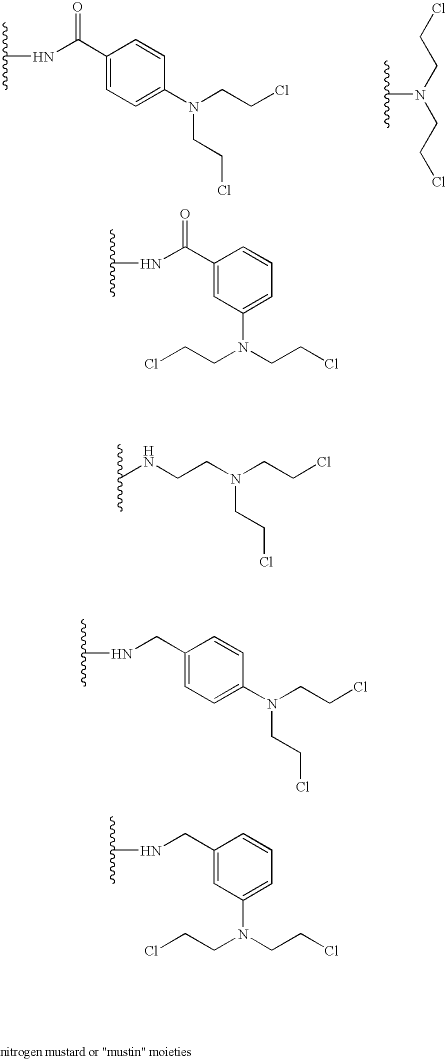

13. (canceled)

14. (canceled)

15. A method of treating or preventing an HSP90-mediated disease, comprising administering to a subject a pharmaceutically effective amount of a composition according to claim 11.

16. A method of treating or preventing an HSP90-mediated disease, comprising administering to a subject a pharmaceutically effective amount of a composition according to claim 8.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. A method of diagnosing or monitoring the progress or regression of an HSP90-mediated disease, comprising:
administering to the cells of a subject having or suspected of having an HSP90-mediated disease a composition according to any one of claims 1, 5-10, or 12; and
evaluating said cells for the presence of said compound.

30. (canceled)

31. (canceled)

32. (canceled)

33. (canceled)

34. (canceled)

35. (canceled)

36. (canceled)

37. (canceled)

38. (canceled)

39. (canceled)
